(19) United States
(12) Patent Application Publication
(10) Pub. No.: US 2021/0369630 A1
(43) Pub. Date: Dec. 2, 2021

(54) MAGNETICALLY RESPONSIVE COMPOSITE MICROPARTICLES FOR TRIGGERED DELIVERY OF BIOLOGICALLY ACTIVE AGENTS

(71) Applicant: UNIVERSITY OF FLORIDA RESEARCH FOUNDATION, Gainesville, FL (US)

(72) Inventors: Jon P. Dobson, Gainesville, FL (US); Peter S. McFetridge, Gainesville, FL (US); Olivia Lanier, Denver, CO (US)

(21) Appl. No.: 17/286,151

(22) PCT Filed: Oct. 17, 2019
(86) PCT No.: PCT/US2019/056740

§ 371 Date: Apr. 16, 2021

Related U.S. Application Data
(60) Provisional application No. 62/746,620, filed on Oct. 17, 2018.

(51) Int. Cl. A61K 9/50; A61K 47/69; A61N 2/00; A61K 35/50; A61K 45/06

(52) U.S. Cl. CPC A61K 9/5094; A61K 47/6923; A61K 45/06; A61K 35/50; A61N 2/00

(57) ABSTRACT

The present disclosure includes composite microparticles for magnetically triggered release of a biologically active agent. Also included are systems including the biocompatible composite microparticles and an alternating current (AC) magnetic field generator to magnetically trigger release of a biologically active agent from the microparticles. The present disclosure further includes methods of delivering a biologically active agent to a patient in vivo using the microparticles and systems of the present disclosure. The present disclosure also includes methods of making biocompatible composite microparticles of the present disclosure for magnetically triggered release of a biologically active agent.

### CROSS-REFERENCE TO RELATED APPLICATIONS

This application claims priority to, and the benefit of, co-pending U.S. provisional application entitled “MAGNETICALLY RESPONSIVE COMPOSITE MICROPARTICLES FOR TRIGGERED DELIVERY OF BIOLOGICALLY ACTIVE AGENTS” having Ser. No. 62/746,620, filed Oct. 17, 2018, which is incorporated by reference in its entirety.

### BACKGROUND

The development of platforms for the controlled release of therapeutic molecules represents an important research focus and therapeutic goal, as controlled release reduces the frequency of administration, minimizes side effects and improves compliance. However, for biological conditions and diseases with a progression that exhibits strong temporal dependence or for conditions that can result in the evolution of tolerance to the therapeutic during continuous exposure, the development of more sophisticated release systems tailored to the needs to the individual and the disease is needed. Thus, delivery platforms with remotely controlled/triggered release mechanisms have been sought. Internal triggers for release (e.g., shifts in biological pH) suffer from the need for high specificity, lack of control over biological conditions, and high variability of such conditions in vivo due to conditions such as illness, environment, and even diet. External triggers for release (e.g., light) also suffer from clinical translation hurdles, such as depth of penetration in tissues as well as the risk of unintended, undesirable, or even toxic effects caused by some external forces. Also, even some triggered delivery mechanisms that have shown limited success with delivery of small molecule therapeutics are not amenable for adaptation for use with biomacromolecules, such as proteins, polynucleotides, and the like.

### SUMMARY

Briefly described, in various aspects, the present disclosure provides composite microparticles for the magnetically triggered release of biologically active agents and methods of making the composite microparticles, systems for targeted delivery and release of biologically active agents in vivo, and methods for targeted delivery and release of biologically active agents to subjects in need of treatment.

Embodiments of composite microparticles of the present disclosure include: a plurality of magnetic nanoparticles; a biologically active agent; and a biocompatible polymer matrix comprising a polymer having a melting point higher than normal body temperature and lower than a deactivation temperature of the biologically active agent, wherein the magnetic nanoparticles and biologically active agent are at least partially encapsulated in the polymer matrix, such that application of an alternating magnetic field induces the magnetic nanoparticles to generate heat effective to melt the polymer matrix, thereby releasing the biologically active agent.

Systems of the present disclosure for targeted delivery and release of biologically active agents in vivo, in embodiments, include: a plurality of biocompatible microparticles, each microparticle comprising a plurality of magnetic nanoparticles, a biologically active agent, and a biocompatible polymer matrix comprising a polymer having a melting point higher than normal body temperature and lower than a deactivation temperature of the biologically active agent, wherein the magnetic nanoparticles and biologically active agent are encapsulated in the polymer matrix; and an alternating current (AC) magnetic field generator, wherein the plurality of microparticles are adapted for delivery to a subject in need of treatment with the biologically active agent and wherein application of an AC magnetic field to a target area of the patient induces the magnetic nanoparticles to generate heat effective to melt the polymer matrix, thereby releasing the biologically active agent to the target area of the subject in vivo.

In embodiments of the present disclosure, methods for targeted delivery and release of biologically active agents to a subject in need of treatment include: delivering to the subject in need of treatment with the biologically active agent a plurality of biocompatible microparticles each microparticle comprising a plurality of magnetic nanoparticles, a biologically active agent, and a biocompatible polymer matrix comprising a polymer having a melting point higher than normal body temperature and lower than a deactivation temperature of the biologically active agent, wherein the magnetic nanoparticles and biologically active agent are encapsulated in the polymer matrix; applying a magnetic field to a target area of the subject to direct the microparticles to the target area; and applying an AC magnetic field to the target area of the subject effective to induce the magnetic nanoparticles to generate heat effective to melt the polymer matrix, thereby releasing the biologically active agent to the target area of the subject.

Methods of making composite microparticles of the present disclosure, in embodiments, include: providing the biologically active agent in an aqueous solvent to provide a first water phase; combining the first water phase with an oil phase comprising the polymer and magnetic nanoparticles in an oil-based solvent to provide a first water in oil mixture and homogenizing to produce a first emulsion; combining the homogenized first emulsion with a second water phase to provide a second mixture and homogenizing the second mixture to produce a second emulsion; and removing solvent from the second emulsion to produce the microparticles.

Other systems, methods, devices, features, and advantages of the devices and methods will be or become apparent to one with skill in the art upon examination of the following drawings and detailed description. It is intended that all such additional systems, methods, devices, features, and advantages be included within this description, be within the scope of the present disclosure, and be protected by the accompanying claims.

### BRIEF DESCRIPTION OF THE DRAWINGS

Further aspects of the present disclosure will be more readily appreciated upon review of the detailed description of its various embodiments, described below, when taken in conjunction with the accompanying drawings. The components in the drawings are not necessarily to scale, emphasis instead being placed upon clearly illustrating the principles of the present disclosure. Moreover, in the drawings, like reference numerals designate corresponding parts throughout the several views.

FIG. 1A illustrates a schematic of a system for magnetically triggered release according to the present disclosure, showing a close-up of a polycaprolactone (PCL) microparticle containing human placental matrix (hPM) and superparamagnetic iron oxide nanoparticles (SPIONs) before (left) and after (right) alternating magnetic field (AMF) exposure. Polymer melts after exposure to AMF due to energy transfer from the field to the SPIONs, heating the SPIONS and melting the PCL resulting in an enhanced release of hPM. Without AMF exposure, the release would occur slowly over months/years after full degradation of PCL by hydrolysis. P=power, μ₀=magnetic permeability of free space, f=frequency, χ=magnetic Susceptibility (out of phase component), H=magnetic field strength. FIG. 1B is a schematic diagram illustrating heat generation of SPIONs via Neel and Brownian relaxation mechanisms. (Reference [45])

FIG. 2 illustrates an SEM image of composite microparticles using final double emulsion synthesis parameters. Scale bar=100 μm.

FIGS. 3A-3C illustrate passive release plots for hPM from composite microparticles (FIG. 3A) and from PCL microparticles without MNPs (FIG. 3B). Release was converted to cumulative percentages for both samples in FIG. 3C. Error bars represent standard deviation (n=3).

FIGS. 4A-4B are graphs illustrating linear fit of release plots after initial burst release for composite microparticles (FIG. 4A) and for PCL particles with no MNPs (FIG. 4B).

FIG. 5 is a graph illustrating passive release of hPM from composite microparticles for 1 day followed by 2-hours triggered release exposed to various conditions (AMF, 72° C. as positive heat control, 37° C. as passive release control). AMF vs. 37° C. release was significantly different using unpaired two-tailed t-test (α=0.05). n=4-6; error bars represent standard deviation. Values corrected for vehicle control particles with no hPM.

FIGS. 6A-6C are SEM images illustrating composite microparticles exposed to various conditions: AMF (FIG. 6A); 37° C. (FIG. 6B); and 72° C. (FIG. 6C). Scale bar=50 μm.

FIG. 7A is a graph illustrating the Ca²⁺ deposition values from cetylpyridinium chloride (CPC) reaction with Alizarin Red S (ARS) in mesenchymal stem cells (MSCs) cultured with different concentrations of human placental proteins (hPM), with error bars representing standard deviation. FIG. 7B is a graph illustrating Calcium deposition by MSCs fed with osteogenic media with different numbers of supplemented doses of hPM determined by ARS staining and CPC reaction.

FIG. 8 is a graph of cell metabolic activity of MSCs exposed to hPM released from difference amounts of composite microparticles as determined by alamar blue (N=3). Error bars represent standard error.

FIG. 9 is a graph illustrating calcium deposition determined by ARS after 14 days for MSCs treated with supplemented osteogenic media. Significant differences between sample (*) and control (0% hPM) determined by unpaired t-test (α=0.05).

FIGS. 10A-C are images illustrating samples 1-3 of PCL microspheres of the present disclosure encapsulating bovine serum albumin (BSA). FIG. 10D is a graph illustrating cumulative passive release of bovine serum albumin (BSA) over time from samples 1 through 3 illustrated in FIGS. 10A-C.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of biochemistry, molecular biology, tissue engineering, biomedical engineering, pharmacology, physics and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

All publications and patents cited in this specification are cited to disclose and describe the methods and/or materials in connection with which the publications are cited. Publications and patents that are incorporated by reference, where noted, are incorporated by reference as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. Any terms not specifically defined within the instant application, including terms of art, are interpreted as would be understood by one of ordinary skill in the relevant art; thus, is not intended for any such terms to be defined by a lexicographical definition in any cited art, whether or not incorporated by reference herein, including but not limited to, published patents and patent applications. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

It must be noted that, as used in the specification and the appended claims, the singular forms “a,” “an,” and “the” include plural referents unless the context clearly dictates otherwise. Thus, for example, reference to “a cell” includes a plurality of cells. In this specification and in the claims that follow, reference will be made to a number of terms that shall be defined to have the following meanings unless a contrary intention is apparent.

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.”

As used herein, the following terms have the meanings ascribed to them unless specified otherwise. In this disclosure, “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. patent law and the term is open-ended, allowing for the presence of more than that which is recited so long as basic or novel characteristics of that which is recited is not changed by the presence of more than that which is recited, but excludes prior art embodiments.

Prior to describing the various embodiments, the following definitions are provided and should be used unless otherwise indicated.

### Definitions

In describing and claiming the disclosed subject matter, the following terminology will be used in accordance with the definitions set forth below.

The terms “nucleic acid” and “polynucleotide” are terms that generally refer to a string of at least two base-sugar-phosphate combinations. As used herein, the terms include deoxyribonucleic acid (DNA) and ribonucleic acid (RNA) and generally refer to any polyribonucleotide or polydeoxyribonucleotide, which may be unmodified RNA or DNA or modified RNA or DNA. RNA may be in the form of a tRNA (transfer RNA), snRNA (small nuclear RNA), rRNA (ribosomal RNA), mRNA (messenger RNA), anti-sense RNA, RNAi (RNA interference construct), siRNA (short interfering RNA), or ribozymes. Thus, for instance, polynucleotides as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. The terms “nucleic acid sequence” and “oligonucleotide” also encompasses a nucleic acid and polynucleotide as defined above.

In addition, polynucleotide as used herein refers to double-stranded regions comprising RNA or DNA or both RNA and DNA. The strands in such regions may be from the same molecule or from different molecules. The regions may include all of one or more of the molecules, but more typically involve only a region of some of the molecules. One of the molecules of a double-helical region often is an oligonucleotide.

It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including simple and complex cells, inter alia. For instance, the term polynucleotide includes DNAs or RNAs as described above that contain one or more modified bases. Thus, DNAs or RNAs comprising unusual bases, such as inosine, or modified bases, such as tritylated bases, to name just two examples, are polynucleotides as the term is used herein. The terms herein also include naturally occurring, chemically modified, and chemically synthesized DNA/RNA.

The term also includes PNAs (peptide nucleic acids), phosphorothioates, and other variants of the phosphate backbone of native nucleic acids. Natural nucleic acids have a phosphate backbone, artificial nucleic acids may contain other types of backbones, but contain the same bases. Thus, DNAs or RNAs with backbones modified for stability or for other reasons are “nucleic acids” or “polynucleotides” as that term is intended herein.

The term “polypeptides” and “protein” include proteins and fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, “isolated” means removed or separated from the native environment. Therefore, isolated DNA can contain both coding (exon) and noncoding regions (introns) of a nucleotide sequence corresponding to a particular gene. An isolated peptide or protein indicates the protein is separated from its natural environment. Isolated nucleotide sequences and/or proteins are not necessarily purified. For instance, an isolated nucleotide or peptide may be included in a crude cellular extract or they may be subjected to additional purification and separation steps.

With respect to nucleotides, “isolated nucleic acid” refers to a nucleic acid with a structure (a) not identical to that of any naturally occurring nucleic acid or (b) not identical to that of any fragment of a naturally occurring genomic nucleic acid spanning more than three separate genes, and includes DNA, RNA, or derivatives or variants thereof. The term covers, for example but not limited to, (a) a DNA which has the sequence of part of a naturally occurring genomic molecule but is not flanked by at least one of the coding sequences that flank that part of the molecule in the genome of the species in which it naturally occurs; (b) a nucleic acid incorporated into a vector or into the genomic nucleic acid of a prokaryote or eukaryote in a manner such that the resulting molecule is not identical to any vector or naturally occurring genomic DNA; (c) a separate molecule such as a cDNA, a genomic fragment, a fragment produced by polymerase chain reaction (PCR), ligase chain reaction (LCR) or chemical synthesis, or a restriction fragment; (d) a recombinant nucleotide sequence that is part of a hybrid gene, e.g., a gene encoding a fusion protein, and (e) a recombinant nucleotide sequence that is part of a hybrid sequence that is not naturally occurring. Isolated nucleic acid molecules of the present disclosure can include, for example, natural allelic variants as well as nucleic acid molecules modified by nucleotide deletions, insertions, inversions, or substitutions.

It is advantageous for some purposes that a nucleotide sequence is in purified form. The term “purified” in reference to nucleic acids and/or proteins represents that the sequence has increased purity relative to the natural environment.

As used herein, the term “genetically engineered,” with respect to a living organism, refers to an organism that has had its genetic makeup directly manipulated by techniques of biotechnology (as opposed to random changes occurring in nature). Genetically engineered organisms can include mutations involving changes only of the genetically engineered organisms' own genetic material as well as mutations involving insertions of exogenous genetic material, such as insertions resulting in either cisgenic (including exogenous genetic material from the same or a closely related organism) or transgenic (including exogenous genetic material from a non-closely related organism) organisms.

As used herein a “mutation” refers to a heritable change in genetic material, which may include alteration of single base pairs of a nucleic acid, or the deletion, insertion, or rearrangement of larger sections of genes or chromosomes. An “engineered mutation” refers to a mutation created by human design (e.g., the mutation did not spontaneously occur by natural causes and/or was the result of intentional human manipulation). A “genetically modified” organism is an organism whose genetic material has been altered by one or more engineered mutations (e.g., human induced mutations).

The terms “treat”, “treating”, and “treatment” are an approach for obtaining beneficial or desired clinical results. Specifically, beneficial or desired clinical results include, but are not limited to, alleviation of symptoms, diminishment of extent of disease, stabilization (e.g., not worsening) of disease, delaying or slowing of disease progression, substantially preventing spread of disease, amelioration or palliation of the disease state, and remission (partial or total) whether detectable or undetectable. In addition, “treat”, “treating”, and “treatment” can also be therapeutic in terms of a partial or complete cure for a disease and/or adverse effect attributable to the disease. As used herein, the terms “prophylactically treat” or “prophylactically treating” refers completely, substantially, or partially preventing a disease/condition or one or more symptoms thereof in a host. Similarly, “delaying the onset of a condition” can also be included in “prophylactically treating”, and refers to the act of increasing the time before the actual onset of a condition in a patient that is predisposed to the condition.

By “administration” is meant introducing a compound of the present disclosure into a subject; it may also refer to the act of providing a composition of the present disclosure to a subject (e.g., by prescribing).

The term “organism,” “subject,” or “host” refers to any living entity in need of treatment, including humans, mammals (e.g., cats, dogs, horses, mice, rats, pigs, hogs, cows, and other cattle), birds (e.g., chickens), and other living species that are in need of treatment. In particular, the term “host” includes humans. As used herein, the term “human host” or “human subject” is generally used to refer to human hosts. In the present disclosure the term “host” typically refers to a human host, so when used alone in the present disclosure, the word “host” refers to a human host unless the context clearly indicates the intent to indicate a non-human host. Hosts that are “predisposed to” condition(s) can be defined as hosts that do not exhibit overt symptoms of one or more of these conditions but that are genetically, physiologically, or otherwise at risk of developing one or more of these conditions.

“Angiogenesis” is a physiological process involving the growth of new blood vessels. Angiogenesis is an important part of biological processes, such as growth and development, wound healing, embryogenesis, and the like. Excessive angiogenesis can occur when diseased cells produce abnormal amounts of angiogenic growth factors, overwhelming the effects of natural angiogenesis inhibitors. Imbalances between the production of angiogenic growth factors and angiogenesis inhibitors can cause improperly regulated growth or suppression of vascular vessels. Angiogenesis-dependent or related diseases result when new blood vessels either grow excessively or insufficiently. The angiogenesis related disease can include diseases such as, but not limited to, cancer, precancerous tissue, tumors, cardiac infarction, and stroke. Excessive angiogenesis can include: cancer, diabetic blindness, age-related macular degeneration, rheumatoid arthritis, psoriasis, and more than 70 other conditions. Insufficient angiogenesis can include: coronary artery disease, stroke, and delayed wound healing, and is also a factor in tissue engineering as discussed in greater detail in the present disclosure.

As used herein, the term “modulate” and/or “modulator” generally refers to the act of directly or indirectly promoting/activating/inducing/increasing or interfering with/inhibiting/decreasing a specific function and/or trait in a cell/organism. In some instances a modulator may increase or decrease a certain activity or function relative to its natural state or relative to the average level of activity that would generally be expected. Modulation includes causing the overexpression or underexpression of a peptide (e.g., by acting to upregulate or downregulate expression of the peptide), or it may directly interact with the subject peptide to increase and/or decrease activity. Modulation also includes causing the increase or decrease of a specific biological activity or biological event, such as angiogenesis or biological events related to angiogenesis

As used herein “upregulate” refers to the act of increasing the expression and/or activity of a protein or other gene product. “Downregulation” refers to decreasing the expression and/or activity of a protein or other gene product.

The term “isolated cell or population of cells” as used herein refers to an isolated cell or plurality of cells excised from a tissue or grown in vitro by tissue culture techniques. The term “a cell or population of cells” may refer to isolated cells as described above or may also refer to cells in vivo in a tissue of an animal or human.

The term “tissue” generally refers to a grouping of cells organized to cooperatively carry out a biological function and/or serve a biological purpose, such as forming all or part of an organ in an organism (e.g., connective tissue, endothelial tissue). While a “tissue” generally includes a grouping of similar cells, or cells of all the same type, a tissue may also include cells of more than one type where the group of cells as a whole serve a common purpose.

As used herein the term “biocompatible” refers to the ability to co-exist with a living biological substance and/or biological system (e.g., a cell, cellular components, living tissue, organ, etc.) without exerting undue stress, toxicity, or adverse effects on the biological substance or system.

The term “bioscaffold” refers to any biocompatible substrate (naturally derived or synthetic) with sufficient structural stability to support the growth of a living biological substance (e.g., living cells). In embodiments of the present disclosure the biocompatible scaffold material is a naturally derived substrate (e.g., procured from a living organism, but that may have undergone additional processing and treatment; or produced from materials derived from a natural source), such as, but not limited to decellularized human umbilical vein scaffolds, In embodiments, the bioscaffolds of the present disclosure have a three-dimensional structure (rather than a planer, 2-dimensional structure) to support three-dimensional growth of living cells.

As used herein, the term “biodegradable” refers to a material that, over time in a natural environment (e.g., within a living organism or living culture), dissolves, deteriorates, or otherwise degrades and loses its structure integrity and ceases to exist in its original structural form. In embodiments of the present disclosure, biodegradable materials dissolve/degrade over a period of time within a host organism.

The term “biocompatible,” with respect to a substance or fluid described herein has the ability to co-exist with and/or within a living biological substance and/or biological system (e.g., a cell, cellular components, living tissue, organ, organism, etc.) without exerting undue stress, toxicity, or adverse effects on the biological substance or system. In embodiments, particularly in vitro embodiments, “biocompatible” indicates that the substance or fluid can be in proximity to or in contact with a biological cell/particle/entity and does not adversely affect the short-term viability or long-term proliferation of such biological cell/particle/entity within a particular time range.

As used herein, the term “engineered” indicates that the engineered object is created and/or altered by man. An engineered object may include naturally derived substances, but the object itself is altered in some way by human intervention and design.

As used herein, the term “biologically active agent” or “bioactive agent” refers to any biomolecule or other compound having or capable of having some activity, use, and/or effect in a biological system (e.g., when administered to a living subject) and/or in relation to another biomolecule. A biologically active agent can include drugs and other therapeutic agents used to treat various conditions in a living subject.

As used herein, the term “biomolecule” is intended to encompass deoxyribonucleic acid (DNA), ribonucleic acid (RNA), nucleotides, oligonucleotides, nucleosides, proteins, peptides, polypeptides, selenoproteins, antibodies, protein complexes, peptide nucleic acids, lipids, carbohydrates, combinations thereof, and the like. In particular, the biomolecule can include, but is not limited to, naturally occurring substances such as polypeptides, polynucleotides, lipids, fatty acids, glycoproteins, carbohydrates, fatty acids, fatty esters, macromolecular polypeptide complexes, complex polypeptide mixtures (e.g., human placental extract and/or other biological extracts), vitamins, co-factors, whole cells, eukaryotic cells, prokaryotic cells, microorganisms, or combinations thereof.

The term “polymer” includes any compound that is made up of two or more monomeric units covalently bonded to each other, where the monomeric units may be the same or different, such that the polymer may be a homopolymer or a heteropolymer. Representative polymers include, but are not limited to, polycaprolactone (PCL), polyvalerolactone (PVL), Poly(acrylic acid, butyl ester) aka PolyButyl Acrylate, poly(ethylene glycol) (PEG), Polyisobutylene, PEG-Distearate MP, Agarose, Poly(azelaic anhydride), Poly(Ethylene adipate) and mixtures thereof and/or co-polymers thereof), nucleic acids and the like, where the polymers may be naturally occurring, non-naturally occurring, or synthetic. The term “bio-polymer” refers to a polymer made of biologically-derived and/or biologically compatible compounds

The term “polymer matrix material” refers herein to various types of semi-solid to solid materials with a gel-like and/or solid consistency and a structure (e.g., crosslinked) capable of holding a three-dimensional structure and of supporting and/or encapsulating materials, such as biologically active agents, magnetic nanoparticles, and the like. The polymer matrix materials of the present disclosure can be made of synthetic and/or naturally derived polymer materials. In embodiments, the polymer matrix material is formed from polymerized and/or crosslinked polymers described herein.

### Discussion

In accordance with the purpose(s) of the present disclosure, as embodied and broadly described herein, embodiments of the present disclosure, in some aspects, relate to composite microparticles for magnetically triggered release of a biologically active agent under an alternating current (AC) magnetic field, systems including the composite microparticles for targeted delivery and release of biologically active agents in vivo, and methods for targeted delivery and release of a biologically active agent to a subject in need of treatment using the composite microparticles of the present disclosure to deliver the biologically active agent to a target area of the subject.

For triggered release systems, the mechanism activating the release can be divided into two general groups: external and internal triggers. Internal triggered release works by releasing therapeutic/active agents upon changes occurring naturally in the body in variables such as pH, glucose and urea concentration. However, such triggers are dependent on biological conditions and thus offer less control. For instance, pH or chemical/protein levels could vary due to illness, environment, or food intake, which may give rise to unanticipated release, and lead to severe consequences. External triggers for triggered release are activated by an energy transfer outside of the body, such as light, temperature, ultrasound, magnetic fields, or electric fields²⁰. However, many of these systems present crippling drawbacks to their translation to the clinic. For example, ultraviolet and infrared light can only penetrate up to 1 cm and 10 cm respectively into living tissue, which are not clinically relevant depths for many applications²¹. Electric fields interact with human tissue, and thus the use of electric fields is not feasible for drug release applications.

Magnetic particles have been investigated for delivery of small molecules, via linkage to magnetic particles or by release from biodegradable beads via magnetic oscillation (see Lanier, et al., Magnetically triggered release of biologics” International Materials Reviews 2018, https://dol.org/10.1080/09506608.2018.1446280, which is hereby incorporated by reference herein). Some of these systems used a Poly(N-isppropylacrylamide) (PNIPAMM) matrix, and as the particles heat, the mesh of the polymer contracts and, in effect, squeezes the drug out through the “mesh.” However, this system does not work for large macromolecules such as proteins. Systems employing the use of magnetic particles for delivery of larger macromolecules, such as proteins, have thus far shown poor results. However, no available technology enables the magnetically triggered release of biological macromolecules, such as proteins, polynucleotides, and complex mixtures of these and other biologically active agents through melting of the polymer matrix by an AMF.

Thus, compositions, systems, and methods of the present disclosure provide composite microparticles with the ability to deliver and trigger release of a payload including a biologically active agent, such as, but not limited to, biomacromolecules and complex mixtures thereof, to a target area of a subject. Triggered delivery and release is achieved by providing composite microparticles including a polymer matrix made of a polymer (e.g., a biocompatible naturally derived and/or synthetic polymer) having a melting point lower than a deactivation temperature of the biologically active agent payload, such that the heat applied to melt the polymer will not denature or otherwise deactivate the biologically active agent. In embodiments, the polymer matrix also has a melting point higher than that of normal body temperature (e.g., about 36 to 37.2° C., or even slightly elevated body temperature, as in when a subject has a fever. (e.g., about 37.2° C. to 41° C.) in order to avoid unintentional activation and release under normal biological conditions or due to unintended activation due to normal fluctuations in body temperature. The compositions, systems, and methods of the present disclosure utilize heat generated by magnetic nanoparticles under exposure to an alternating magnetic field (AMF) to trigger melting of the polymer matrix and release of a payload including a biologically active agent.

In embodiments, the composite microparticles of the present disclosure include a plurality of magnetic nanoparticles and a biologically active agent at least partially encapsulated in a polymer matrix including a polymer having a melting point higher than normal body temperature (including the range of biologically survivable, elevated body temperature) and lower than a deactivation temperature of the biologically active agent, such that application of an alternating magnetic field induces the magnetic nanoparticles to generate heat effective to melt the polymer matrix, thereby releasing the biologically active agent. While some biologically active agents, e.g., DNA and some proteins begin to denature at temperatures of about 41° C., it may take prolonged exposure at these temperatures and many can withstand temperatures above this as well.

In embodiments, the polymer matrix has a melting temperature of about 38° C. or higher (e.g., 70° C. In embodiments, the melting temperature can range from about 40° C. to 65° C.; in embodiments the melting temperature is from about 42° C. to 60° C. In embodiments, the polymer matrix is made of a polymer material having a melting temperature of about 42° C. to 64.5° C. (determined by differential scanning calorimetry).

In embodiments, the polymer matrix includes one or more biocompatible polymers from the group including, but not limited to, polycaprolactone (PCL), polyvalerolactone (PVL), Poly(acrylic acid, butyl ester) aka PolyButyl Acrylate, PEG4000, and PEG6000, Polyisobutylene, PEG-Distearate MP, Agarose, Poly(azelaic anhydride), Poly(Ethylene adipate). In embodiments, the polymer has a molecular weight ranging from about 1000 Da to 100,000 Da. In embodiments, the polymer matrix is made of PCL (e.g., PCL having a molecular weight of about 1200 Da, 14,000 Da, 45,000 Da, 60,000 Da, 75,000 Da, 80,000 Da, 90,000 Da as well as other molecular weights). In embodiments, the polymer matrix material cross links or otherwise interacts with itself or other materials to encapsulate the biologically active agent and the magnetic nanoparticles.

In embodiments, the magnetic nanoparticles are made of iron oxides, iron oxyhydroxides, cobalt ferrites, iron-nickel, or other magnetic materials. In embodiments, the magnetic nanoparticles are made of biocompatible materials or are coated with a biocompatible coating/surfactant to provide/improve biocompatibility. In embodiments, the magnetic nanoparticles are superparamagnetic iron oxide nanoparticles (SPIONs) made of one or more iron oxides, such as Fe₃O₄, γFe₂O₃, or combinations thereof. In embodiments, the magnetic nanoparticles have an average diameter of about 5 nm to 100 nm (this “core diameter” is the diameter of the magnetic particle without any coating, as determined by transmission electron microscopy). Commercially available magnetic particles often come with a thin polymer coating giving them a “hydrodynamic diameter” (core diameter plus polymer coating) of about 20 nm to 300 nm as determined by nanoparticle tracking analysis (NTA) and/or dynamic light scattering (DLS) (see Table 3.1, below). Magnetic particles can also be described in term s of a “magnetic diameter,” which is determined by fitting magnetization curves (determined by vibrating sample magnetometry) to the Langevin-Chantrell function. In embodiments, the magnetic nanoparticles are SPIONs having an average magnetic diameter of about 20 nm or less. In embodiments the composite microparticles include about 1% (w/w) magnetic nanoparticles, or more. In embodiments, the microparticles include about 5% (w/w) magnetic nanoparticles or more, such as about 5 to 80% weight magnetic nanoparticles.

In embodiments, the composite microparticles can have an average diameter of about 1 to 100 μm (determined by light microscopy). In embodiments, the composite microparticles have an average diameter of about 1-10 μm. In embodiments, the microparticles include about 0.25 to 40% (w/w) biologically active agent. In embodiments, the microparticles have a loading of about 10 to 50%, or more, biologically active agent.

The disclosed composite microparticles can be used to deliver any payload to a subject in vivo (e.g., to tissues, vessels, cells, tumors, implanted tissue scaffolds, etc.). In embodiments of the present disclosure, the payload can be a biologically active agent, such as a biomolecule, small molecule, and other therapeutic and/or diagnostic agents. In embodiments, the payload is a biomolecule or combination of biomolecules. In embodiments, the biomolecule or combination thereof, includes biomacromolecules such as proteins, protein complexes (protein matrices or mixed protein extracts, polynucleotides (e.g., DNA, RNA, etc.). In embodiments, the biologically active agent payload is a protein extract that includes a complex mixture of proteins (e.g., a crude protein extract or a more purified extract with some components removed). In embodiments, the payload is a complex human placental extract/matrix (hPM) having a combination of multiple cytokines and other growth factors and other placental proteins. In embodiments, the biologically active agent (e.g., “payload”) is an hPM described in Moore, M. C., Pandolfi, V. & McFetridge, P. S. Novel human-derived extracellular matrix induces in vitro and in vivo vascularization and inhibits fibrosis. Biomaterials 49, 37-46 (2015), which is hereby incorporated by reference herein.

In embodiments, the payload can include polynucleotides and may be employed for gene therapy or other genetic treatment of a subject. In embodiments, the payload can include a combination of proteins, polynucleotides, other biomolecules (e.g., carbohydrates, lipids, etc.) as well as small molecules (e.g., therapeutics, etc.).

In some embodiments, the payload can include an anti-cancer agent that can cause apoptosis, pyroptosis, or necrosis of a targeted tumor cell. In some embodiments, the anti-cancer agent is a small molecule drug. In some embodiments, the anti-cancer agent is monomethyl auristatin E, gemcitabine, or resveratrol.

In embodiments, the biologically active agent can further include one or more of classes of antibiotics (e.g., Aminoglycosides, Cephalosporins, Chloramphenicol, Clindamycin, Erythromycins, Fluoroquinolones, Macrolides, Azolides, Metronidazole, Penicillins, Tetracyclines, Trimethoprim-sulfamethoxazole, Vancomycin). The provided composition(s) can further include one or more of classes of steroids (e.g., Andranes (e.g., Testosterone). The payload can further include one or more of classes of narcotic and non-narcotic analgesics (e.g., Morphine, Codeine, Heroin, Hydromorphone, Levorphanol, Meperidine, Methadone, Oxydone, Propoxyphene, Fentanyl, Methadone, Naloxone, Buprenorphine, Butorphanol, Nalbuphine, Pentazocine).

The payload can further include one or more of classes of anti-inflammatory agents (e.g., Alclofenac, Alclometasone Dipropionate, Algestone Acetonide, alpha Amylase, Amcinafal, Amcinafide, Amfenac Sodium, Amiprilose Hydrochloride, Anakinra, Anirolac, Anitrazafen, Apazone, Balsalazide Disodium, Bendazac, Benoxaprofen, Benzydamine Hydrochloride, Bromelains, Broperamole, Budesonide, Carprofen, Cicloprofen, Cintazone, Cliprofen, Clobetasol Propionate, Clobetasone Butyrate, Clopirac, Cloticasone Propionate, Cormethasone Acetate, Cortodoxone, Decanoate, Deflazacort, Delatestryl, Depo-Testosterone, Desonide, Desoximetasone, Dexamethasone Dipropionate, Diclofenac Potassium, Diclofenac Sodium, Diflorasone Diacetate, Diflumidone Sodium, Diflunisal, Difluprednate, Diftalone, Dimethyl Sulfoxide, Drocinonide, Endrysone, Enlimomab, Enolicam Sodium, Epirizole, Etodolac, Etofenamate, Felbinac, Fenamole, Fenbufen, Fenclofenac, Fenclorac, Fendosal, Fenpipalone, Fentiazac, Flazalone, Fluazacort, Flufenamic Acid, Flumizole, Flunisolide Acetate, Flunixin, Flunixin Meglumine, Fluocortin Butyl, Fluorometholone Acetate, Fluquazone, Flurbiprofen, Fluretofen, Fluticasone Propionate, Furaprofen, Furobufen, Halcinonide, Halobetasol Propionate, Halopredone Acetate, Ibufenac, Ibuprofen, Ibuprofen Aluminum, Ibuprofen Piconol, Ilonidap, Indomethacin, Indomethacin Sodium, Indoprofen, Indoxole, Intrazole, Isoflupredone Acetate, Isoxepac, Isoxicam, Ketoprofen, Lofemizole Hydrochloride, Lomoxicam, Loteprednol Etabonate, Meclofenamate Sodium, Meclofenamic Acid, Meclorisone Dibutyrate, Mefenamic Acid, Mesalamine, Meseclazone, Mesterolone, Methandrostenolone, Methenolone, Methenolone Acetate, Methylprednisolone Suleptanate, Momiflumate, Nabumetone, Nandrolone, Naproxen, Naproxen Sodium, Naproxol, Nimazone, Olsalazine Sodium, Orgotein, Orpanoxin, Oxandrolane, Oxaprozin, Oxyphenbutazone, Oxymetholone, Paranyline Hydrochloride, Pentosan Polysulfate Sodium, Phenbutazone Sodium Glycerate, Pirfenidone, Piroxicam, Piroxicam Cinnamate, Piroxicam Olamine, Pirprofen, Prednazate, Prifelone, Prodolic Acid, Proquazone, Proxazole, Proxazole Citrate, Rimexolone, Romazarit, Salcolex, Salnacedin, Salsalate, Sanguinarium Chloride, Seclazone, Sermetacin, Stanozolol, Sudoxicam, Sulindac, Suprofen, Talmetacin, Talniflumate, Talosalate, Tebufelone, Tenidap, Tenidap Sodium, Tenoxicam, Tesicam, Tesimide, Testosterone, Testosterone Blends, Tetrydamine, Tiopinac, Tixocortol Pivalate, Tolmetin, Tolmetin Sodium, Triclonide, Triflumidate, Zidometacin, Zomepirac Sodium).

The payload can further include one or more of classes of anti-histaminic agents (e.g., Ethanolamines (like diphenhydrmine carbinoxamine), Ethylenediamine (like tripelennamine pyrilamine), Alkylamine (like chlorpheniramine, dexchlorpheniramine, brompheniramine, triprolidine), other anti-histamines like astemizole, loratadine, fexofenadine, Bropheniramine, Clemastine, Acetaminophen, Pseudoephedrine, Triprolidine).

Numerous anti-cancer (antineoplastic) drugs are available for combination with the present method and compositions. Antineoplastic drugs include Acivicin, Aclarubicin, Acodazole Hydrochloride, AcrQnine, Adozelesin, Aldesleukin, Altretamine, Ambomycin, Ametantrone Acetate, Aminoglutethimide, Amsacrine, Anastrozole, Anthramycin, Asparaginase, Asperlin, Azacitidine, Azetepa, Azotomycin, Batimastat, Benzodepa, Bicalutamide, Bisantrene Hydrochloride, Bisnafide Dimesylate, Bizelesin, Bleomycin Sulfate, Brequinar Sodium, Bropirimine, Busulfan, Cactinomycin, Calusterone, Caracemide, Carbetimer, Carboplatin, Carmustine, Carubicin Hydrochloride, Carzelesin, Cedefingol, Chlorambucil, Cirolemycin, Cisplatin, Cladribine, Crisnatol Mesylate, Cyclophosphamide, Cytarabine, Dacarbazine, Dactinomycin, Daunorubicin Hydrochloride, Decitabine, Dexormaplatin, Dezaguanine, Dezaguanine Mesylate, Diaziquone, Docetaxel, Doxorubicin, Doxorubicin Hydrochloride, Droloxifene, Droloxifene Citrate, Dromostanolone Propionate, Duazomycin, Edatrexate, Eflomithine Hydrochloride, Elsamitrucin, Enloplatin, Enpromate, Epipropidine, Epirubicin Hydrochloride, Erbulozole, Esorubicin Hydrochloride, Estramustine, Estramustine Phosphate Sodium, Etanidazole, Ethiodized Oil I 131, Etoposide, Etoposide Phosphate, Etoprine, Fadrozole Hydrochloride, Fazarabine, Fenretinide, Floxuridine, Fludarabine Phosphate, Fluorouracil, Flurocitabine, Fosquidone, Fostriecin Sodium, Gemcitabine, Gemcitabine Hydrochloride, Gold Au 198, Hydroxyurea, Idarubicin Hydrochloride, Ifosfamide, Ilmofosine, Interferon Alfa-2a, Interferon Alfa-2b, Interferon Alfa-n1, Interferon Alfa-n3, Interferon Beta-I a, Interferon Gamma-Ib, Iproplatin, Irinotecan Hydrochloride, Lanreotide Acetate, Letrozole, Leuprolide Acetate, Liarozole Hydrochloride, Lometrexol Sodium, Lomustine, Losoxantrone Hydrochloride, Masoprocol, Maytansine, Mechlorethamine Hydrochloride, Megestrol Acetate, Melengestrol Acetate, Melphalan, Menogaril, Mercaptopurine, Methotrexate, Methotrexate Sodium, Metoprine, Meturedepa, Mitindomide, Mitocarcin, Mitocromin, Mitogillin, Mitomalcin, Mitomycin, Mitosper, Mitotane, Mitoxantrone Hydrochloride, Mycophenolic Acid, Nocodazole, Nogalamycin, Ormaplatin, Oxisuran, Paclitaxel, Pegaspargase, Peliomycin, Pentamustine, Peplomycin Sulfate, Perfosfamide, Pipobroman, Piposulfan, Piroxantrone Hydrochloride, Plicamycin, Plomestane, Porfimer Sodium, Porfiromycin, Prednimustine, Procarbazine Hydrochloride, Puromycin, Puromycin Hydrochloride, Pyrazofurin, Riboprine, Rogletimide, Safmgol, Safingol Hydrochloride, Semustine, Simtrazene, Sparfosate Sodium, Sparsomycin, Spirogermanium Hydrochloride, Spiromustine, Spiroplatin, Streptonigrin, Streptozocin, Strontium Chloride Sr 89, Sulofenur, Talisomycin, Taxane, Taxoid, Tecogalan Sodium, Tegafur, Teloxantrone Hydrochloride, Temoporfin, Teniposide, Teroxirone, Testolactone, Thiamiprine, Thioguanine, Thiotepa, Tiazofurin, Tirapazamine, Topotecan Hydrochloride, Toremifene Citrate, Trestolone Acetate, Triciribine Phosphate, Trimetrexate, Trimetrexate Glucuronate, Triptorelin, Tubulozole Hydrochloride, Uracil Mustard, Uredepa, Vapreotide, Verteporfin, Vinblastine Sulfate, Vincristine Sulfate, Vindesine, Vindesine Sulfate, Vinepidine Sulfate, Vinglycinate Sulfate, Vinleurosine Sulfate, Vinorelbine Tartrate, Vinrosidine Sulfate, Vinzolidine Sulfate, Vorozole, Zeniplatin, Zinostatin, Zorubicin Hydrochloride.

Other anti-neoplastic compounds include: 20-epi-1,25 dihydroxyvitamin D3, 5-ethynyluracil, abiraterone, aclarubicin, acylfulvene, adecypenol, adozelesin, aldesleukin, ALL-TK antagonists, altretamine, ambamustine, amidox, amifostine, aminolevulinic acid, amrubicin, atrsacrine, anagrelide, anastrozole, andrographolide, angiogenesis inhibitors, antagonist D, antagonist G, antarelix, anti-dorsalizing morphogenetic protein-1, antiandrogen, prostatic carcinoma, antiestrogen, antineoplaston, antisense oligonucleotides, aphidicolin glycinate, apoptosis gene modulators, apoptosis regulators, apurinic acid, ara-CDP-DL-PTBA, arginine deaminase, asulacrine, atamestane, atrimustine, axinastatin 1, axinastatin 2, axinastatin 3, azasetron, azatoxin, azatyrosine, baccatin III derivatives, balanol, batimastat, BCR/ABL antagonists, benzochlorins, benzoylstaurosporine, beta lactam derivatives, beta-alethine, betaclamycin B, betulinic acid, bFGF inhibitor, bicalutamide, bisantrene, bisaziridinylspermine, bisnafide, bistratene A, bizelesin, breflate, bropirimine, budotitane, buthionine sulfoximine, calcipotriol, calphostin C, camptothecin derivatives, canarypox IL-2, capecitabine, carboxamide-amino-triazole, carboxyamidotriazole, CaRest M3, CARN 700, cartilage derived inhibitor, carzelesin, casein kinase inhibitors (ICOS), castanospermine, cecropin B, cetrorelix, chlorines, chloroquinoxaline sulfonamide, cicaprost, cis-porphyrin, cladribine, clomifene analogues, clotrimazole, collismycin A, collismycin B, combretastatin A4, combretastatin analogue, conagenin, crambescidin 816, crisnatol, cryptophycin 8, cryptophycin A derivatives, curacin A, cyclopentanthraquinones, cycloplatam, cypemycin, cytarabine ocfosfate, cytolytic factor, cytostatin, dacliximab, decitabine, dehydrodidemnin B, deslorelin, dexifosfamide, dexrazoxane, dexverapamil, diaziquone, didemnin B, didox, diethylnorspermine, dihydro-5-azacytidine, dihydrotaxol, 9-dioxamycin, diphenyl spiromustine, docosanol, dolasetron, doxifluridine, droloxifene, dronabinol, duocannycin SA, ebselen, ecomustine, edelfosine, edrecolomab, eflornithine, elemene, emitefur, epirubicin, epristeride, estramustine analogue, estrogen agonists, estrogen antagonists, etanidazole, etoposide phosphate, exemestane, fadrozole, fazarabine, fenretinide, filgrastim, fmasteride, flavopiridol, flezelastine, fluasterone, fludarabine, fluorodaunorunicin hydrochloride, forfenimex, formestane, fostriecin, fotemustine, gadolinium texaphyrin, gallium nitrate, galocitabine, ganirelix, gelatinase inhibitors, gemcitabine, glutathione inhibitors, hepsulfam, heregulin, hexamethylene bisacetamide, hypericin, ibandronic acid, idarubicin, idoxifene, idramantone, ilmofosine, ilomastat, imidazoacridones, imiquimod, immunostimulant peptides, insulin-like growth factor-1 receptor inhibitor, interferon agonists, interferons, interleukins, iobenguane, iododoxorubicin, ipomeanol, 4-irinotecan, iroplact, irsogladine, isobengazole, isohomohalicondrin B, itasetron, jasplakinolide, kahalalide F, lamellarin-N triacetate, lanreotide, leinamycin, lenograstim, lentinan sulfate, leptolstatin, letrozole, leukemia inhibiting factor, leukocyte alpha interferon, leuprolide+estrogen+progesterone, leuprorelin, levamisole, liarozole, linear polyamine analogue, lipophilic disaccharide peptide, lipophilic platinum compounds, lissoclinamide 7, lobaplatin, lombricine, lometrexol, lonidamine, losoxantrone, lovastatin, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, menogaril, merbarone, meterelin, methioninase, metoclopramide, MIF inhibitor, mifepristone, miltefosine, mirimostim, mismatched double stranded RNA, mitoguazone, mitolactol, mitomycin analogues, mitonafide, mitotoxin fibroblast growth factor-saporin, mitoxantrone, mofarotene, molgramostim, monoclonal antibody, human chorionic gonadotrophin, monophosphoryl lipid A+myobacterium cell wall sk, mopidamol, multiple drug resistance genie inhibitor, multiple tumor suppressor 1-based therapy, mustard anticancer agent, mycaperoxide B, mycobacterial cell wall extract, myriaporone, N-acetyldinaline, N-substituted benzamides, nafarelin, nagrestip, naloxone+pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, O6-benzylguanine, octreotide, okicenone, oligonucleotides, onapristone, ondansetron, ondansetron, oracin, oral cytokine inducer, ormaplatin, osaterone, oxaliplatin, oxaunomycin, paclitaxel analogues, paclitaxel derivatives, palauamine, palmitoylrhizoxin, pamidronic acid, panaxytriol, panomifene, parabactin, pazelliptine, pegaspargase, peldesine, pentosan polysulfate sodium, pentostatin, pentrozole, perflubron, perfosfamide, perillyl alcohol, phenazinomycin, phenylacetate, phosphatase inhibitors, picibanil, pilocarpine hydrochloride, pirarubicin, piritrexim, placetin A, placetin B, plasminogen activator inhibitor, platinum complex, platinum compounds, platinum-triamine complex, porfimer sodium, porfiromycin, propyl bis-acridone, prostaglandin J2, proteasome inhibitors, protein A-based immune modulator, protein kinase C inhibitor, protein kinase C inhibitors, microalgal, protein tyrosine phosphatase inhibitors, purine nucleoside phosphorylase inhibitors, purpurins, pyrazoloacridine, pyridoxylated hemoglobin polyoxyethylene conjugate, raf antagonists, raltitrexed, ramosetron, ras farnesyl protein transferase inhibitors, ras inhibitors, ras-GAP inhibitor, retelliptine demethylated, rhenium Re 186 etidronate, rhizoxin, ribozymes, RII retinamide, rogletimide, rohitukine, romurtide, roquinimex, rubiginone B1, ruboxyl, safingol, saintopin, SarCNU, sarcophytol A, sargramostim, Sdi 1 mimetics, semustine, senescence derived inhibitor 1, sense oligonucleotides, signal transduction inhibitors, signal transduction modulators, single chain antigen binding protein, sizofiran, sobuzoxane, sodium borocaptate, sodium phenylacetate, solverol, somatomedin binding protein, sonermin, sparfosic acid, spicamycin D, spiromustine, splenopentin, spongistatin 1, squalamine, stem cell inhibitor, stem-cell division inhibitors, stipiamide, stromelysin inhibitors, sulfmosine, superactive vasoactive intestinal peptide antagonist, suradista, suramin, swainsonine, synthetic glycosaminoglycans, tallimustine, tamoxifen methiodide, tauromustine, tazarotene, tecogalan sodium, tegafur, tellurapyrylium, telomerase inhibitors, temoporfin, temozolomide, teniposide, tetrachlorodecaoxide, tetrazomine, thaliblastine, thalidomide, thiocoraline, thrombopoietin, thrombopoietin mimetic, thymalfasin, thymopoietin receptor agonist, thymotrinan, thyroid stimulating hormone, tin ethyl etiopurpurin, tirapazamine, titanocene dichloride, topotecan, topsentin, toremifene, totipotent stem cell factor, translation inhibitors, tretinoin, triacetyluridine, triciribine, trimetrexate, triptorelin, tropisetron, turosteride, tyrosine kinase inhibitors, tyrphostins, UBC inhibitors, ubenimex, urogenital sinus-derived growth inhibitory factor, urokinase receptor antagonists, vapreotide, variolin B, vector system, erythrocyte gene therapy, velaresol, veramine, verdins, verteporfin, vinorelbine, vinxaltine, vitaxin, vorozole, zanoterone, zeniplatin, zilascorb, zinostatin stimalamer.

The herein provide composition can further comprise one or more additional radiosensitizers. Examples of known radiosensitizers include gemcitabine, 5-fluorouracil, pentoxifylline, and vinorelbine. (Zhang et al., 1998; Lawrence et al., 2001; Robinson and Shewach, 2001; Strunz et al., 2002; Collis et al., 2003; Zhang et al., 2004).

In embodiments, payload can include a combination of any one or more of the above biologically active agents (e.g., a protein extract and a therapeutic agent (e.g., a chemotherapy agent) as well as a biologically active imaging agent (e.g., for imaging of the microparticles in vivo (in addition to the magnetic nanoparticles)) or a targeted delivery probe (e.g., for directing the magnetic particles to a specific organ/tissue/target area, etc.).

Embodiments of the present disclosure also include systems for imaging and targeted delivery and release of a biologically active agent(s) in vivo using the composite microparticles of the present disclosure. In embodiments, systems of the present disclosure include a plurality of biocompatible microparticles of the present disclosure, where each microparticle includes a plurality of magnetic nanoparticles, a biologically active agent, and a polymer matrix, as described above. In embodiments, the polymer has a melting point higher than normal body temperature and lower than a deactivation temperature of the biologically active agent (e.g., human placental extract). In embodiments, the plurality of microparticles are adapted for delivery to a subject in need of treatment with the biologically active agent, and where application of an AC magnetic field to a target area of the patient induces the magnetic nanoparticles to generate heat effective to melt the polymer matrix, thereby releasing the biologically active agent to the target area of the subject in vivo.

The present disclosure also induces methods of treatment with the composite microspheres of the present disclosure and a magnetic field. Embodiments of methods of treatment of the present disclosure include methods of targeted delivery and release of a biologically active agent to a subject in need of treatment. Embodiments of such methods include delivering to the subject in need of treatment with the biologically active agent a plurality of microparticles of the present disclosure as described above, where each microparticle includes a plurality of magnetic nanoparticles, a biologically active agent, and a polymer matrix made of at least one polymer having a melting point higher than normal body temperature and lower than a deactivation temperature of the biologically active agent, where the magnetic nanoparticles and biologically active agent are encapsulated in the polymer matrix. Then, the patient is subject to a magnetic field to activate/trigger direct delivery of the therapeutic to the target area of a subject. In embodiments the target area may be a specific tissue type, an organ, an injury site or wound, a site of inflammation, damage, infection or other disorder.

Methods of the present disclosure also include applying an alternating magnetic field (AMF) to a target area of the subject, where the AMF is effective to induce the magnetic nanoparticles to generate heat effective to melt the polymer matrix, thereby releasing payload including the biologically active agent to the target area of the subject. In embodiments an AC magnetic field has a field strength of about 5 to 50 kA/m and frequencies of about 100 to 1,000 kHz. In embodiments the AC magnetic field is applied for a time period of about 0.1 to 3 hours. In embodiments, the AC magnetic field is applied for about 1 to 2 hours.

Methods of the present disclosure also include methods of making the composite microparticles of the present disclosure described above. In embodiments, the microparticles can be made using a double water in oil in water emulsion. In an embodiment of a method of making composite microparticles of the present disclosure, the method includes providing the biologically active agent in an aqueous solvent/carrier to provide a first water phase, providing the polymer and magnetic nanoparticles in an oil based solvent/carrier (oil phase), and providing a second aqueous carrier/solvent as the second water phase, with an optional emulsifier. In embodiments an emulsifier can be included in the first water phase, the second water phase, or both. In embodiments the emulsifying agent is selected from sucrose, polyethylene glycol (PEG), pluronic, tween, and polyvinyl alcohol (PVA). In embodiments, the emulsifier is from about 0.05%-5% by weight of the second water phase.

In embodiments, the first water phase (including the biologically active agent) can be combined/mixed into the first oil phase and mixed (e.g., homogenized, sonicated, vortexed, or otherwise mixed) to produce a first water in oil mixture/emulsion. Then the first mixture/emulsion can be combined with the second water phase to provide a second mixture and mixed/homogenized to produce a second emulsion. In embodiments, the first mixture is homogenized for about 30 seconds to 4 minutes and the second mixture is homogenized for about 30 seconds to 4 minutes. Modifications to homogenization time can be used to adjust the size of the resulting microparticles.

After homogenization, the solvent can be removed from the second emulsion (e.g., via evaporation, filtration, centrifugation, other methods or combination of methods) to produce the microparticles. After separation of the microparticles by removing solvent from the emulsion, the microparticles can be washed and stored. In embodiments, the separated microparticles can be lyophilized to produce freeze-dried microparticles that can be stored for later use with hydration. In embodiments lyophilized microparticles are stored at a temperature of about 4° C.

Also, variations in the amounts of solvent, magnetic nanoparticles, polymer, emulsifier, biologically active agent, etc. can be used to adjust the resulting particle size and parameters such as loading of magnetic nanoparticles, loading of biological active agent, melting time, and other features of the composite microparticles. For instance, polymer concentration in the solvent can range from about 1% to 40%, and increasing polymer concentration will result in larger particles with higher loading efficiencies of magnetic particles and therapeutic. The volume ratio of the first water phase to the oil phase can be varied between about 1% and 20%, and increasing the ratio will result in a lower loading efficiency of therapeutic. The percentage of emulsifying agent in the first and second water phases can be varied between about 0.05% and 5%, and increasing this percentage will result in smaller particle sizes and higher loading efficiency. The amount of magnetic particles in the oil phase can be varied from about 0.05% to 20% w/w, and increasing this percentage will decrease the loading efficiency. Further, the ratio of the first emulsion (first water phase and oil phase) to the second water phase can be varied from about 5% to 50%, and increasing this ratio will result in larger particles. Additional details regarding the methods for making embodiments of composite microparticles of the present disclosure are provided in Examples 1 and 2 below.

Additional details regarding the methods, compositions, and organisms of the present disclosure are provided in the Examples below. The specific examples below are to be construed as merely illustrative, and not limitative of the remainder of the disclosure in any way whatsoever. Without further elaboration, it is believed that one skilled in the art can, based on the description herein, utilize the present disclosure to its fullest extent.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and protected by the following claims.

The following examples are put forth so as to provide those of ordinary skill in the art with a complete disclosure and description of how to perform the methods and use the compositions and compounds disclosed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to 5%” should be interpreted to include not only the explicitly recited concentration of about 0.1 wt % to 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value. In addition, the phrase “about ‘x’ to ‘y’” includes “about ‘x’ to about ‘y’”.

### Aspects

The following listing of exemplary aspects supports and is supported by the disclosure provided herein.

Aspect 1. A composite microparticle comprising:
a plurality of magnetic nanoparticles;
a biologically active agent; and
a biocompatible polymer matrix comprising a polymer having a melting point higher than normal body temperature and lower than a deactivation temperature of the biologically active agent, wherein the magnetic nanoparticles and biologically active agent are at least partially encapsulated in the polymer matrix, such that application of an alternating magnetic field induces the magnetic nanoparticles to generate heat effective to melt the polymer matrix, thereby releasing the biologically active agent.

Aspect 2. The composite microparticle of aspect 1, wherein the magnetic nanoparticles comprise superparamagnetic iron oxide nanoparticles (SPIONs).

Aspect 3. The composite microparticle of aspect 2, wherein the SPIONs are selected from the group consisting of: Fe₃O₄ and γFe₂O₃.

Aspect 4. The composite microparticle of any of aspects 1-3, wherein the magnetic nanoparticles/SPIONs have an average diameter of about 5 nm to 100 nm.

Aspect 5. The composite microparticle of any of aspects 1-4, wherein the biologically active agent comprises biomolecules.

Aspect 6. The composite microparticle of aspect 5, wherein the biomolecules comprise proteins, antibodies, nucleotides, carbohydrates, lipids, and mixtures thereof.

Aspect 7. The composite microparticle of any of aspects 1-6, wherein the biologically active agent comprises human placental matrix (hPM).

Aspect 8. The composite microparticle of any of aspects 1-7, wherein the biologically active agent comprises small molecule therapeutic agents.

Aspect 9. The composite microparticle of aspect 8, wherein the small molecule therapeutic agents are selected from the group consisting of: drugs, anti-cancer agents, antibiotics, anti-neoplastics, anti-inflammatory agents, pain management agents, and combinations thereof.

Aspect 10. The composite microparticle of any of aspects 1-9, wherein the polymer matrix comprises a biocompatible polymer having a melting point of about 38° C. or higher.

Aspect 11. The composite microparticle of any of aspects 1-9, wherein the polymer matrix comprises a biocompatible polymer having a melting point of about 42° C. to 60° C.

Aspect 12. The composite microparticle of any of aspects 1-11, wherein the polymer matrix comprises a biocompatible polymer selected from the group consisting of: polycaprolactone (PCL), polyvalerolactone (PVL), Poly(acrylic acid, butyl ester) aka PolyButyl Acrylate, PEG4000, and PEG6000, Polyisobutylene, PEG-Distearate MP, Agarose, Poly(azelaic anhydride), Poly(Ethylene adipate), and combinations thereof.

Aspect 13. The composite microparticle of aspect 12, wherein the polymer matrix comprises PCL having a molecular weight from about 1200 Da to 80,000 Da.

Aspect 14. The composite microparticle of any of aspects 1-13, wherein the microparticles comprise about 10 to 50% biologically active agent.

Aspect 15. The composite microparticle of any of aspects 1-14, wherein the microparticles comprise about 5 to 80% (w/w) magnetic nanoparticles.

Aspect 16. The composite microparticle of any of aspects 1-15, wherein the microparticles are produced by a double water/oil/water emulsion.

Aspect 17. The composite microparticle of any of aspects 1-16, wherein the microparticles are lyophilized.

Aspect 18. A system for targeted delivery and release of biologically active agents in vivo, the system comprising:
a plurality of biocompatible microparticles, each microparticle comprising a plurality of magnetic nanoparticles, a biologically active agent, and a biocompatible polymer matrix comprising a polymer having a melting point higher than normal body temperature and lower than a deactivation temperature of the biologically active agent, wherein the magnetic nanoparticles and biologically active agent are encapsulated in the polymer matrix; and
an alternating current (AC) magnetic field generator, wherein the plurality of microparticles are adapted for delivery to a subject in need of treatment with the biologically active agent and wherein application of an AC magnetic field to a target area of the patient induces the magnetic nanoparticles to generate heat effective to melt the polymer matrix, thereby releasing the biologically active agent to the target area of the subject in vivo.

Aspect 19. The system of aspect 18, wherein the field generator has a field strength of about 5 to 50 kA/m.

Aspect 20. The system of any of aspects 18-19, wherein the field generator has a frequency of about 100 to 1,000 kHz.

Aspect 21. The system of any of aspects 18-20, wherein the plurality of biocompatible microparticles do not substantially release the biologically active agent until application of the AC magnetic field.

Aspect 22. A method for targeted delivery and release of a biologically active agent to a subject in need of treatment, the method comprising:
delivering to the subject in need of treatment with the biologically active agent a plurality of biocompatible microparticles each microparticle comprising a plurality of magnetic nanoparticles, a biologically active agent, and a biocompatible polymer matrix comprising a polymer having a melting point higher than normal body temperature and lower than a deactivation temperature of the biologically active agent, wherein the magnetic nanoparticles and biologically active agent are encapsulated in the polymer matrix;
applying a magnetic field to a target area of the subject to direct the microparticles to the target area; and
applying an AC magnetic field to the target area of the subject effective to induce the magnetic nanoparticles to generate heat effective to melt the polymer matrix, thereby releasing the biologically active agent to the target area of the subject.

Aspect 23. The method of aspect 22, wherein the AC magnetic field has a strength of about 5 to 37.5 kA/m.

Aspect 24. The method of any of aspects 22-23, wherein the AC magnetic field frequency of about 100 to 1,000 kHz.

Aspect 25. The method of any of aspects 22-24, wherein the AC magnetic field is applied for a time period of about 0.1 to 3 hours.

Aspect 26. The method of any of aspects 22-25, wherein the plurality of microparticles do not substantially release the biologically active agent until application of the AC magnetic field.

Aspect 27. A method of making composite microparticles of any of aspects 1-17, the method comprising:
providing the biologically active agent in an aqueous solvent to provide a first water phase;
combining the first water phase with an oil phase comprising the polymer and magnetic nanoparticles in an oil-based solvent to provide a first water in oil mixture and homogenizing to produce a first emulsion;
combining the homogenized first emulsion with a second water phase to provide a second mixture and homogenizing the second mixture to produce a second emulsion; and
removing solvent from the second emulsion to produce the microparticles.

Aspect 28. The method of aspect 27, wherein the polymer concentration in the oil phase is from about 1% to 40% (w/w).

Aspect 29. The method of any of aspects 27-28, wherein the concentration of magnetic particles in the oil phase is from about 0.05% to 20% (w/w).

Aspect 30. The method of any of aspects 27-29, wherein a volume ratio of the first water phase to the oil phase is about 1% to 20%.

Aspect 31. The method of any of aspects 27-30, wherein a volume ratio of the first emulsion to the second water phase is from about 5% to 50%.

Aspect 32. The method of any of aspects 27-31, further comprising adding an emulsifying agent to the first water phase, the second water phase, or both.

Aspect 33. The method of aspect 32, wherein the amount of emulsifying agent in either the first, second or both water phases is from about 0.05% to 5% (w/w).

Aspect 34. The method of any of aspects 33-34, wherein the emulsifying agent is selected from surcrose, PEG, and PVA.

Aspect 35. The method of any of aspects 27-35, further comprising lyophilizing the microparticles to produce freeze dried microparticles.

From the foregoing, it will be seen that aspects herein are well adapted to attain the ends and objectives hereinabove set forth together with other advantages which are obvious and which are inherent to the systems and methods.

It will be understood that certain features and subcombinations are of utility and may be employed without reference to other features and subcombinations. This is contemplated by and is within the scope of the aspects.

While specific elements and steps are discussed in connection to one another, it is understood that any element and/or steps provided herein is contemplated as being combinable with any other elements and/or steps regardless of explicit provision of the same while still being within the scope provided herein. Since many possible aspects may be made of the disclosure without departing from the scope thereof, it is to be understood that all matter herein set forth or shown in the accompanying drawings is to be interpreted as illustrative and not in a limiting sense

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

The following examples describe a non-invasive system with dynamic control over delivery of complex biologically active agents that allows adjusting release to the patient's changing physiological requirements. The system described in the Example 1 includes polycaprolactone microparticles encapsulating hPM and superparamagnetic iron oxide nanoparticles (SPIONs). Heat generated by SPIONs exposed to an alternating magnetic field (AMF) will melt the PCL, thus changing the diffusion characteristics of hPM through the polymer. Example 2 describes methods for making and varying different properties of microparticles of the present disclosure. In Example 3, below, SPIONs with high heat generation in an AMF were identified and characterized.

### Acronyms in the Following Examples

Human placental matrix (hPM); Polycaprolactone (PCL); Superparamagnetic iron oxide nanoparticles (SPIONs); Alternating magnetic field (AMF); American Academy of Orthopaedic Surgeons (AAOS); Centers for Disease Control and Prevention (CDC); Insulin-like growth factor-binding protein 1 (IGFBP-1); neutrophil-activating peptide-2 (NAP-2); Extracellular matrix (ECM); Defensin alpha 1 (DEFA1); Human umbilical vein endothelial cells (HUVEC); Transforming growth factor beta 1 (TGFB1); Vascular endothelial growth factor A (VEGFA); Poly(lactic-co-glycolic acid) (PLGA); Poly(lactic acid) (PLA); Poly(glycolic acid) (PGA); Poly(ethylene glycol) (PEG); Food and Drug Administration (FDA); Superparamagnetism (SPM); Specific absorption rate (SAR); Intrinsic loss parameter (ILP); Nanoparticle tracking analysis (NTA); Dynamic light scattering (DLS); Ultraviolet-visible (UV-Vis); Bovine serum albumin (BSA); Nerve growth factor (NGF); water1/oil/water2 (W1/O/W2); Poly(vinyl alcohol) (PVA); Phosphate buffered saline (PBS); Bicinchoninic acid Protein Assay (BCA Protein Assay); Differential scanning calorimetry (DSC); Dynamic magnetic susceptibility (DMS); Carboxylic acid (COOH); Amine (NH₂); Polyethylenimine (PEI)

### Example 1: Magnetically Responsive Composite Microparticles for the Triggered Delivery of Human Placental Matrix (hPM) a

### General Discussion

Vascular insufficiency is a significant obstacle to the innate healing process, and thus can cause complication in the healing of chronic injuries, nonunion fractures, and myocardial tissue after infarction. Blood vessel growth requires a complex array of growth factors and signaling molecules working synergistically, and recent attempts at regenerating tissues through the delivery of one or two growth factors have been met with limited success in clinical trials². The McFetridge lab has successfully shown induction of capillary beds in vitro and in vivo using a novel matrix derived from the human placenta—the human placental matrix (hPM)³. hPM is composed of a complex mixture of cytokines, growth factors, chemokines, and extracellular matrix proteins and is hypothesized to better model the natural angiogenic processes, as compared to doses of a single growth factor (see Moore, et al. 2015, and U.S. Pat. No. 9,821,013, which are incorporated herein by reference). However, the vascular networks induced by a single dose of hPM deteriorate over time, indicating the need for an improved delivery technique.

Twenty million people worldwide are affected annually by a loss of bone tissue due to trauma or disease. The field of tissue engineering has seen the creation of scaffolds which mimic the extracellular matrix to provide structural support aiding in the formation of new bone. However, control of cell activity within the scaffold has not been fully achieved in these systems. Next-generation scaffolds attempt to further improve tissue regeneration through utilizing microparticles for the local delivery of bioactive molecules. Yet these typically only aim to deliver 1-2 growth factors. For more comprehensive bone regeneration, the system of the present example uses a complex mixture of proteins derived from human placental tissues. The placental proteins have been shown to induce angiogenesis and osteogenesis, but these responses have been shown to deteriorate with lack of continued dosing.

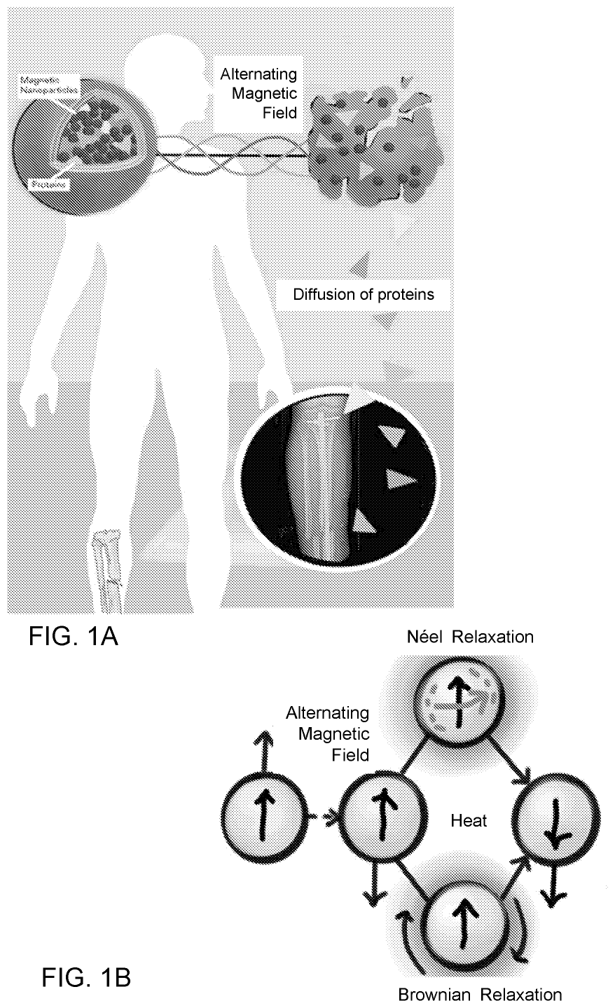

Therefore, this example describes a non-invasive, remotely controlled delivery platform capable of administering multiple doses of hPM, as required. This delivery platform will include polycaprolactone (PCL) composite microparticles encapsulating superparamagnetic iron oxide nanoparticles (SPIONs) and hPM. Release of hPM cargo from composite microparticles can be remotely controlled and enhanced due to melting of the PCL (Tₘ˜60° C.) achieved through heat generation by SPIONs exposed to an alternating magnetic field (AMF) as illustrated in FIG. 1A. The ability to trigger the release of complex protein mixtures on-demand will provide a significant advantage with wounds such as large segmented bone defects where stagnation of the healing process can occur.

This example describes initial development of a model and a delivery platform for hPM tailored to the patient's changing physiological needs. Potential clinical applications include conditions such as managing severe extremity trauma in combat soldiers and civilians and preventing both fracture nonunion and infarction due to coronary attack. This approach of hPM delivery for enhanced wound healing is innovative at least because magnetically triggered release of protein is scarce in the literature, and has never been designed for delivery of multiple proteins at once; it takes into account various parameters that can influence the kinetic release; and it allows for the design of a system capable of multiple cycles of release. The model described in this example can be used to optimize this delivery platform for other drugs/biologics with minimal experimental studies, and will show how changing magnetic field properties will affect release profiles. The system can also be expanded for other clinically relevant delivery platforms such as, but not limited to, sustained and controlled delivery of insulin in patients with diabetes. Additionally, this work provides a solid framework and fundamental understanding for magnetically triggered release of other drugs/biologics that can be applied in alternative engineering areas as well, such as water purification and agriculture.

Angiogenesis and Human Placental Matrix (hPM)

Coronary heart disease is responsible for 1 in 6 deaths in the United States, and approximately 1,450,000 coronary attacks occurred in 2010 in the United States alone⁴. Approximately 7,900,000 million bone fractures occur in the United States per year, and 5 to 10% of these have delayed healing⁵; Specifically, nonunion is the failure of innate healing of a bone fracture. In fact, 20 million people annually are affected worldwide by a loss of bone tissue due to trauma or disease. The likelihood of a delayed union or nonunion is increased with severity of trauma, age, smoking, and underlying metabolic conditions such as diabetes.

Thus, there is a clinical need for alternative bone repair strategies. To address this need, the field of tissue engineering has seen the creation of scaffolds made from metals, ceramics, and polymers which mimic the extracellular matrix (ECM) to provide structural support aiding in the formation of new bone. There are specific concerns that must be addressed when designing a bone scaffold including biocompatibility, biodegradability, mechanical durability, appropriate porosity (200 to 500 μm is considered ideal for bone regeneration), proper nano/micro-topography, and sterilibility; however, control of cell activity, especially differentiation, within the scaffold has not been fully achieved in these systems. To address this, next generation bone tissue engineering scaffolds incorporate bioactive molecules involved in natural bone healing processes into the scaffold to promote more robust regeneration. Different biomolecules, growth factors, etc. are activated at different phases of bone healing, and it is a complex process difficult to replicate. However, there have been efforts to incorporate various of the biomolecules implicated in bone healing into engineered scaffolds for bone and tissue repair by covalent binding, physical entrapment, and through use of controlled delivery particles. Examples of growth factor delivery platforms for bone regeneration are reviewed in De Witte et al.; however most engineered scaffolds typically only deliver 1-2 growth factors rather than the complex mixture of proteins that are involved in the various phases of the natural healing process.

Angiogenesis is an essential part of healing, and has been implicated as a potential therapy for both coronary disease and fractures nonunion. The capillary networks formed during angiogenesis deliver nutrients and oxygen to the injury site to facilitate healing. Angiogenesis is a spatiotemporal process involving many growth factors, cytokines, and chemokines. Previous methods employed to induce angiogenesis include delivery of single or basic combinations of growth factors, genes which result in the production of angiogenic growth factors, stem cells, and microRNA. These efforts for angiogenesis induction have been met with limited success for differing reasons. Delivery of single or basic combinations of growth factors lacks the broad spectrum of cytokines and chemical gradients involved in in vivo healing and have short circulation times. Gene delivery lacks the necessary efficiency, and has been the subject of serious safety concerns. Stem cell delivery can be immunogenic and requires large numbers of cells, and microRNA therapies lack the necessary understanding for clinical translation due to their complex regulatory actions.

In pursuit of a solution to this ongoing problem, a human placental matrix (hPM) has been developed as described in Moore et al. (2015) and Tonello, et al. (2016) (both of which are hereby incorporated by reference herein). hPM is a solution that can contain over 2600 proteins derived from human placental tissues. Among these proteins are angiogenic cytokines: IGFBP-1, NAP-2, and angiogenin; ECM components: fibrinogen, laminin; and immune-related cytokines: annexins, and DEFA1. In human umbilical vein endothelial cells (HUVECs), hPM was shown to induce vasculogenesis and upregulate angiogenic genes including TGFB1 and VEGFA. In vivo, hPM was shown to induce angiogenesis within bioengineered scaffolds after 5 days. Scaffolds dosed with hPM showed minimal fibrosis compared to control scaffolds, which is hypothesized to be due to the presence of anti-inflammatory immune proteins in hPM.

In addition, hPM has been shown to stimulate chondrogenesis in vitro (Wable, D. et al. Effects of the Human Placental Matrix and Culture Conditions on Chondrogenesis in Mesenchymal Stem Cells. in BMES Annual Meeting (2018)). MSCs fed with chondrogenic media supplemented with 50% hPM for 7 days showed increased staining for cartilage (Safranin O), collagen (Sirius Red), and glycosaminoglycans (Alcian blue)²²¹. The current study has also shown hPM to stimulate osteogenesis in vitro.

Unfortunately, the angiogenic networks induced by a single dose of hPM can deteriorate within a week after a single dose, indicating the need for sustained delivery. Poly(lactic-co-glycolic acid) (PLGA) microparticles were synthesized for the controlled release of hPM. HUVECs were cultured in 3D alginate hydrogels embedded with the hPM loaded PLGA microparticles, and the induced capillary networks were shown to remain viable over 28 days (see Tonello, et al. (2016) as well as U.S. Pat. No. 10,300,091, which are incorporated by reference herein]. This provided proof of concept that controlled delivery is possible for hPM. Yet, for in vivo wound healing, a temporally dynamic controlled release system will better facilitate long term maintenance of vascular networks. This will be particularly important for traumatic injuries, such as the ones described above, which require extended healing times (e.g. up to nine months after surgical treatment of a tibial nonunion) and may require multiple burst releases of hPM in addition to passive release achieved by a basic controlled release system.

### Magnetically Triggered Delivery Platform

The success of the systems and methods of the present disclosure lies not only in the use of hPM to trigger angiogenesis, but also in the unique and non-invasive delivery platform which will allow for the sustained and triggered delivery of hPM. Remote control over the release kinetics of the hPM will be useful for patients with changing physiological requirements that may need numerous bursts of hPM in order to achieve proper healing. Triggered delivery involves a controlled release system that sustains or holds release until activated by an internal or external trigger, at which point it will undergo an increased burst release. The embodiment of this delivery platform described in this example include PCL composite microparticles encapsulating magnetic nanoparticles (MNPs) and hPM with an applied AMF as the triggering mechanism (FIG. 1A).

Controlled and Triggered Release

For the proposed application of delivering hPM for induction of angiogenesis, controlled release is not sufficient, as multiple burst releases may be needed in order to elicit a proper healing response. There are numerous other applications where remote control over drug release is desired including biological conditions and diseases with a progression that exhibit strong temporal dependency, or those that can result in the evolution of tolerance to the therapeutic during continuous exposure.

External triggers for triggered release can include events induced by a force outside of the body, such as light, temperature, ultrasound, magnetic fields, or electric fields. Many of these systems have crippling drawbacks to their translation to the clinic. For example, ultraviolet and infrared light can only penetrate up to 1 cm and 10 cm respectively into living tissue, which are not clinically relevant depths for many applications. Electric fields interact with human tissue, and thus the use of electric fields is not feasible for drug release applications. Internal triggered release is a self-regulated system that works by releasing drug upon changes in variables such as pH, glucose, or urea concentration. Internal triggers require high specificity, and pH or chemical/protein levels could vary due to illness, environment, or food intake, which may give rise to unanticipated release, and lead to severe consequences.

On the other hand, magnetically triggered release is an attractive option because (i) AMFs used to trigger release are able to penetrate completely through human tissue; (ii) the SPIONs used are minimally toxic and can be metabolized by the body; and (iii) it gives the user complete external control over the system. Magnetically triggered release utilizes magnetic particles as the remote control modality to trigger delivery from a carrier vesicle, such as a polymer microsphere or liposome, encapsulating the drug. AMFs transfer energy to the magnetic particles, causing them to heat, and subsequently enhance the release of the drug/biologic as illustrated in FIG. 1.

For the design of this platform, many criteria were considered. The system is able to maintain therapeutic levels for an extended duration, maintain functionality of the hPM proteins, biodegrade, not induce an immune response, be scalable and sterilizable, and respond to the heat produced by MNPs in an AMF.

Polycaprolactone (PCL)

Polycaprolactone (PCL) (structure shown), a hydrophobic, semi-crystalline polymer, was chosen as the material of choice for this example embodiment of the present disclosure for numerous reasons. Importantly, it has a relatively low melting temperature, 59°-64° C.²⁵, which will enable the heat generation from SPIONs in an AMF to melt it and change the diffusivity characteristics without deactivating any biological materials, such as proteins, contained in the microparticles. PCL microspheres (Mₙ ˜43-50 k) were recently used in a study for magnetically triggered release of the small molecule antibiotic ciprofloxacin²⁶. PCL been used in long term protein delivery systems, including delivery systems that require release longer than one year. However, due to its low melting temperature, PCL cannot be heat sterilized and often PCL products must be made using aseptic technique after filter sterilization of solubilized PCL in organic solvent

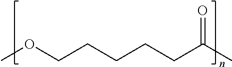

PCL is biodegradable via surface or bulk hydrolysis depending on the formulation, and it achieves bioresorption after 24 months²⁵. Due to its slow degradation, release of proteins from PCL is typically diffusion controlled³¹. In addition, PCL is less likely to generate an acidic environment upon degradation than poly(lactic acid) (PLA) and poly(glycolic acid) (PGA)³², which is important for the proposed application because an acidic environment could adversely affect the hPM activity.

PCL and PCL copolymers have low toxicity²⁵. For that reason and the reasons detailed above, PCL and PCL copolymers have been employed in multiple clinical applications. PCL is used in the implantable one year contraceptive release device, Capronor™, in the USA and Europe. In Europe, it is used as a degradable staple, and a PGA/PCL copolymer is used as a suture marketed as Monocyrl™. It is also used in block copolymers with PGA, PLA, and poly(ethylene glycol) (PEG) for SynBiosys™, a commercially available drug delivery device²⁵. Additionally, it is used in Resilon™, a root canal filling product³⁰. The use of PCL in already clinically used devices reduces the potential regulatory barriers in achieving FDA approval for the proposed technology.

Superparamagnetic Iron Oxide Nanoparticles (SPIONs)

This Example utilizes heat generation by SPIONs exposed to an AMF to trigger PCL melting and hPM release. Sigma Aldrich 900042 MNPs were used as they were identified in Example 3, below, to have one of the highest heating efficiencies, quantified by specific absorption rate (SAR) out of a panel of 31 commercially available MNPs. Sigma Aldrich 900042 MNPs are made of iron oxide and coated with 2,5 Furandione polymer with methoxyethene. Iron oxide has low toxicity compared to other magnetic materials and can be metabolized by the body.

In order to understand how this heat generation is possible, it is important to first understand the magnetic properties of iron oxide, and how these properties change at the nanoscale. Magnetic properties in materials arise due to both the orbiting motion and spin of the electrons. Magnetite and maghemite are ferrimagnetic iron oxide materials, which means these material contains uncompensated electron spins that are coupled unequally over space, and it responds positively to an external magnetic field. At the bulk scale, ferrimagnetic materials contain multiple domains within which the electron spins are aligned, but the domains are not necessarily aligned with each other. The domains exist to reduce magnetostatic energy associated with stray fields, and when an external magnetic field is applied, the material will expend energy to grow the domains in the direction of the field at the expense of those in different directions. Yet, when shrunk to the nanoscale, ferromagnetic materials (as well as ferromagnets and antiferromagnets) will become single-domain because the energy required to create a domain wall exceeds the energy required to support the magnetostatic energy of a single domain³³. In a single domain particle, the magnetic moment of the entire particle points in one direction, either parallel or antiparallel with the easy axis of magnetization.

Superparamagnetism (SPM), a phenomenon arising in single-domain magnetic materials, is dependent on material composition, size, temperature, and measurement time. This phenomenon is governed by the Néel-Arrhenius equation:

$$τ = {τ}_{0} exp ( \frac{KV}{{k}_{B} T} ) \qquad ( 1 )$$

where τ is the relaxation time of the net magnetization of the particle, τ₀ is the attempt time, K is the anisotropy energy density, V is the volume of the magnetic particle, and k_{B}T is the thermal energy³⁴. In zero magnetic field, thermal energy exceeds the magnetic energy for SPM particles at room temperature, causing the magnetic moment to flip between energy minima, i.e. parallel or antiparallel with the easy axis of magnetization. This flipping occurs so rapidly that even over measurement times on the order of seconds to minutes, the particles have zero net remanent magnetization. Thus, unless extremely close together, SPM particles do not recognize each other as magnets in the absence of a field, especially when coated. When a magnetic field is applied, the magnetic moment of SPM particles will align in the direction of the field, and the particles elicit a strong, positive response to the magnetic field. When the field is removed, the SPM particles exhibit no remnant magnetization and no hysteresis. For iron oxide materials, SPM properties are observed at diameters of 20 nm or less, depending on particle shapes³⁵. Magnetite, in particular, has a very strong shape anisotropy that enables smaller particles to appear magnetically blocked.

Due to their superparamagnetic properties, SPIONs have been researched extensively for use in biomedical applications. After coating with a biocompatible polymer, SPIONs are able to be dispersed throughout the blood with reduced likelihood of aggregation due to magnetic dipole interactions, which could cause harmful clotting of the vasculature³⁶. An added benefit of SPIONs over SPM particles synthesized from other materials, such as cobalt ferrites, is that they are naturally metabolized by the body³⁷. Applications in which SPIONs have been used in the clinic or in biomedical research include: magnetic resonance imaging contrast agents, drug targeting to a particular area in the body³⁸, magnetic fluid hyperthermia for tumor destruction³⁹, magnetically guided lesion localization³⁸, ion channel opening⁴¹, non-viral gene transfection⁴², blood filtration⁴³, triggered drug delivery⁴⁴, and cell separation immunoassays.

When placed in an alternating magnetic field, magnetic particles will generate heat due to two mechanisms (FIG. 1B). The first, Brownian relaxation, is the physical rotation of magnetic particles in response to the fluctuating field that creates frictional energy as heat, and is dependent on fluid properties. The second is Néel relaxation, in which the magnetic moment attempts to flip but cannot keep up with the speed of the field and the phase lag is dissipated as heat (see Pankurst, et al., Applications of magnetic nanoparticles in biomedicine,” J. of Physics D: Applied Physics, v. 36, No. 13, 2003). These mechanisms are both characterized by relaxation times that vary depending on particle and field properties.

Power generated by SPM particles in an AMF is defined by⁴⁶:

P_{SPM}=μ₀πƒχ″H² (2)

where μ₀ is the permeability of free space, f is the frequency, χ″ is the out-of-phase component of the complex susceptibility χ=χ′+iχ″, and H is the magnetic field strength⁴⁶. The heating potential of magnetic particles is typically reported as the specific absorption rate (SAR) in units W/g⁴⁷:

$$SAR = \frac{Δ T}{Δ t} * \frac{C}{{m}_{Fe}} \qquad ( 3 )$$

where C is the heat capacity of fluid per unit mass of fluid and m_{Fe} is the iron mass in the fluid per unit mass of fluid. In order to account for discrepancies in SARs measured in magnetic fields with varying properties, the intrinsic loss parameter (ILP) was introduced with units of nHm²/kg⁴⁸:

$$ILP = \frac{SAR}{{H}^{2} f} \qquad ( 4 )$$

Heat generated due to AMFs has been shown to positively correlate with increased magnetic field strength and frequency⁴⁸. However, if the AMF is too strong, it has the potential to cause non-specific heating in the human body through eddy currents. Therefore, the “sweet spot” for field conditions is desired, and in general magnetic field strengths of 0-15 kA/m and frequencies of 0.05-1.2 MHz can be used safely²³.

### Materials and Methods

Materials for Particle Synthesis

Chemicals used for particle synthesis include: Oleic acid (Sigma Aldrich 364525); Dimethyl sulfoxide (Fisher Chemical D128-1); Chloroform (Fisher Chemical C298-4); Polyvinyl Alcohol M_{w} 31,000-50,000 (Sigma Aldrich 363073); Polycaprolactone Mₙ 1,040 kDa (Lactel); Polycaprolactone M_{w} 14,000 (Simga Aldrich 440752); Polycaprolactone Mₙ 45,000 (Sigma Aldrich 704105); Polycaprolactone Mₙ 80,000 (Sigma Aldrich 440744). Chemicals used for particle hydrolysis include sodium hydroxide (Fisher Scientific S318-1); sodium doedecyl sulfate (MP Biomedicals, Fisher Scientific 190522); and hydrochloric acid (Sigma Aldrich 320331).

Theoretical Determination of Optimal Particle Size

Preliminary calculations show that it is feasible to melt PCL microspheres (M_{w}˜14,000) if the microspheres are 5% (w/w) MNPs. Sigma Aldrich MNPs (Product #900042) were used for this calculation as they had the highest SAR value of measured MNPs when exposed to a magnetic field of strength 26 kA/m with a frequency of 223 kHz (see Chapter 3).

The power generated from the MNPs was estimated using the SAR by BLM, 1083.8±102.4 W/g MNP. The energy required to melt the PCL microspheres was taken as the latent heat of melting from the DSC data. In the case of PCL microspheres (M_{w}˜14,000) with encapsulated protein, 110.8 J/g of PCL.

An energy balance can then be used to solve for the time required to melt the PCL. Note that this assumes 5% (w/w) MNPs.

$$108 \; 3.8 \frac{W}{g MNP} * 0.05 {m}_{microsphere} * time = 110.8 \frac{J}{g} * 0.95 {m}_{microsphere} \\ time = 1.94 seconds \qquad ( 4 \text{-} 1 )$$

where m_{microsphere} is the mass of microsphere.

Preliminary mass transfer calculations were performed to determine if diffusion of hPM through melted PCL is different than through solid PCL microspheres. Time for diffusivity was estimated by²³¹:

$${τ}_{d} = \frac{{R}^{2}}{D} \qquad ( 4 \text{-} 2 )$$

where R is the hydrodynamic radius of the microsphere, and D is the diffusivity of protein. The diffusivity coefficient for protein from melted polymer was estimated using the Stokes-Einstein Equation^{232,233}:

$$D = \frac{{k}_{B} T}{6 π η R} \qquad ( 4 \text{-} 3 )$$

where k_{B} is Boltzmann's constant, T is temperature, η is the dynamic viscosity of fluid or melted polymer, and Rₚᵣₒₜₑᵢₙ is the radius of the diffusing protein. By dividing D of the protein through melted polymer by D of the protein through water, the equation can be simplified in order to estimate the value of D for protein through melted polymer:

$$\frac{D , melted PCL}{D , water} = \frac{η , water}{η , melted PCL} \qquad ( 4 \text{-} 4 )$$

D of protein through water was found to be approximately 10⁻¹⁰ m²/s in literature²³⁴. The viscosity of water²³⁵ is 1.12*10⁻³ Pa*s and the viscosity of melted PCL²³⁶ is 100 Pa*s. Therefore, the value of D through melted PCL was estimated to be 1.12*10⁻¹⁵ m²/s. Putting this value back into the time for diffusivity equation (4-3), the radius for a diffusion time on the timescale of minutes was estimated. With a radius of 50 μm, it would take the protein ˜26 days to diffuse through the polymer melt, and with a radius of 1 micron the diffusion time is only ˜15 minutes. It would be ideal to synthesize microparticles with a hydrodynamic diameter radius which is able to retain encapsulated hPM until magnetically triggered, at which point it would need ˜15 minutes to release the entire encapsulated payload. These calculations are only rough estimates, but show that microparticles with relatively small diameters 1-10 μm would be preferable, which is important for the design.

Coating MNPs with Oleic Acid

1 mg of MNPs (Sigma Aldrich 900042) was separated from water by centrifugation (Eppendorf, model 5415D) for 40 min at 15,000 rcf in a 1.5 ml tube (Eppendorf 05-408-129). The water is removed from the MNP pellet and then the MNP pellet is suspended in 337 μL oleic acid and 6 ml of dimethyl sulfoxide in a Pyrex tube. The solution is then heated on a magnetic hot plate set to 300° C. for 6 minutes. The solution is then vortexed (Vortex Genie 2; Scientific Industries) and transferred to a 50 ml centrifuge tube (Corning 430828). The solution is magnetically separated overnight and washed with 10 ml DMSO. Then the DMSO was magnetically separated overnight and the next day the MNPs suspended in 10 ml chloroform for use in the double emulsion for microparticle synthesis.

Synthesis of Composite Microparticles Via Double Emulsion

PCL composite microparticles were synthesized via a water/oil/water double emulsion (W1/O/W2) followed by lyophilization. Briefly, 1000 mg PCL was dissolved in 20 ml chloroform (O) containing 10 mg oleic acid coated 900042 MNPs. 3 ml hPM (W1) was added to O and vortexed for 30 s and then homogenized for 4 min at 12,000 rpm (Scilogex D160 homogenizer 85010301). Then, W1/O was added to W2, which consists of 25 ml 0.5% poly(vinyl alcohol) (PVA) dissolved in water, and subsequently homogenized for 2 min at 8,000 rpm. W1/O/W2 was stirred overnight using an orbital shaker plate (Lab-Line Instruments, model 4626) at 225 rpm to allow for solvent evaporation. Then, microparticles were centrifuged (Thermo Fisher Scientific, Forma 400 ml GP) in 50 ml tube (Corning 430828) s for 5 min at 1500 rpm, washed with distilled water three times, transferred to 2 ml cryotubes (Corning 430569) and lyophilized (Labconco, Freezone 4.5). The dry microparticles were stored at 4° C.

Double emulsion parameters such as amount of polymer used, O to W1 volume ratio, W1/O to W2 volume ratio, protein concentration in W1, PVA concentration in W2, molecular weight of polymer, primary and secondary emulsion homogenization times, evaporation time, number of washes, and mechanism of stirring for evaporation were varied in original experiments in order to find synthesis parameters that resulted in microparticles with the correct size and relatively high protein/MNP loading.

Vibrating Sample Magnetometry for Determination of MNP Content in Composite Microparticles

MNP loading was analyzed using vibrating sample magnetometry as described above with 5-10 mg of composite microparticles loaded into the alginate gels. The loading percentage was calculated using the following equation:

$$Loading Percentage = \frac{{X}_{i} - {X}_{f}}{{X}_{i}} * 100 \% \qquad ( 4 \text{-} 5 )$$

where Xᵢ is the initial amount of MNPs that was attempted to be loaded and X_{ƒ} is the actual amount of MNPs loaded.

Quantification of Protein Loading in Composite Microparticles

First, the protein content in hPM was evaluated for (n=27) using the Pierce BCA protein detection kit (Thermo Fisher Scientific) in order to calculate the theoretical amount of protein loaded into microparticles. Protein loading was measured by three different methods: (1) indirect protein loading; (2) dissolution of PCL microparticles; and (3) NaOH hydrolysis. Indirect protein loading was performed by quantifying the amount of protein in the wash supernatants collected during microparticle synthesis using the Pierce BCA Protein Assay Kit (Thermo Fisher Scientific), thus measuring the amount of protein that was not loaded into the microparticles. Dissolution was performed by suspending 30-50 mg of composite microparticles in 4 ml chloroform or dichloromethane and mixed for 40 min under magnetic stirring in a Pyrex tube (10 ml). Then, 2 ml PBS was added and vigorously mixed for 24 hr. The mixture was transferred to a 1.5 ml centrifuge tube (Eppendorf 05-408-129) and centrifuged (Eppendorf, Centrifuge 5415D) for 10 min at 13,000 rpm. The aqueous phase supernatant was collected and assayed for protein content using the Micro BCA Protein Assay Kit (Thermo Fisher Scientific). Finally, for the NaOH hydrolysis method, 15 mg composite microparticles were weighed into 15 ml centrifuge tubes (Falcon 352099) and 5 ml of 0.5 M NaOH plus 5% SDS was added. The mixture was hydrolyzed for 72 hours and then neutralized using HCl and NaOH until the pH was between 6-8. Then, the solution was assayed for protein content using the Micro BCA Protein Assay Kit (Thermo Fisher Scientific).

The loading percentage of proteins within the microparticles was calculated as in Equation 4-5. As a control for all three protein detection methods, vehicle microparticles containing only MNPs and no protein were synthesized and their loading signal was subtracted from the loading signal of composite microparticles with MNPs and hPM.

Differential Scanning Calorimetry to Determine Melting Point of Composite Microparticles

Composite microparticles made from various molecular weights of PCL were evaluated for melting temperature using a Differential Scanning Calorimeter (Instrument Specialists Inc., Model DSC 550). The temperature was varied between 0° C. to 70° C. for two cycles at a rate of 10° C./min.

Scanning Electron Microscopy Imaging of Composite Microparticles

Scanning electron microscopy (SEM, Thermo Fisher Scientific, Phenom Pro Desktop SEM) with a CeB₆ gun source at 5 kV was used to analyze composite microparticles after treatment to evaluate signs of polymer melting. For SEM analysis, particle samples were mounted on a metallic stub with carbon tape. Excess particles were removed by blowing the samples with air. The particle samples were then coated with carbon using a Hummer 6.2 sputter coater (Anatech USA) in order to reduce accumulation of negative charge in the SEM.

Light Microscopy for Composite Microparticle Size Determination

Light microscopy (Thermo Fisher EVOS FL) was used for sizing composite microparticles. For imaging the microparticles, between 2.5 mg and 5 mg of composite microparticles was weighed into 1.5 ml microcentrifuge tubes (Fisherbrand 05-408-129) using an analytical balance (Mettler Toledo AL104). 1 ml of distilled water was pipetted into each tube and each tube was then vortexed for 10 s. To image a sample, 40 μl of the microparticle solution was pipetted onto the glass microplate slides. 10 images were taken of each sample at 20× magnification, making sure to avoid imaging the same particle twice.

Experiments for Protein Release from Composite Microparticles

100 mg of composite microparticles were suspended in 0.6 ml of distilled water in 1.5 ml centrifuge tubes (Fisherbrand 05-408-129). The samples were vortexed for 30 s to suspend the microparticles in solution. The samples were incubated at 37° C. on an incubator shaker (Thermo Scientific MaxQ 4450) at 150 rpm. At specified time points, the samples were centrifuged for 10 minutes at 16,100 rcf and supernatants collected. The microparticles were then suspended in fresh distilled water. Supernatants were assayed for protein content using the micro BCA protein detection kit (ThermoFisher Scientific 23235). Before being assayed, supernatants were filtered with 0.45 μm filters (Fisherbrand 09-720-4) to remove residual particles from solution that may interfere with the absorbance assay. Composite microparticles with no loaded protein were ran as a control and their signal subtracted from the signal of the protein loaded microparticles. Microparticles with hPM and no MNPs were also evaluated for release to determine the effect of the MNPs on the release profile.

For triggered release experiments, the samples were passively released for 24 hours at 37° C. on the incubator shaker. Then, the supernatants were collected and fresh distilled water added to the sample. The samples were vortexed and transferred to 8 well strips (Corning 9102) for easy placement into the center of the AMF coil and subjected to one of the following conditions for 1 hours or 2 hours: 37° C. on incubator shaker, 72° C. on incubator shaker, AMF. AMFs were applied using an Ambrell Easyheat™ induction heating systems which include the induction heating power supply and work head as described in Section 3.2.1. A solenoid coil is attached to the work head which is connected to the induction heater power supply. A pump and heat exchanger pumped cooling water through the system at set point 20° C. The coil used (horizontal; length=0.135 m; diameter=0.045 m; n-turns=14) operates at 300.3 A and 223 kHz, with a measured field strength of 51 mT.

After applying the condition, the samples were transferred back to 1.5 ml centrifuge tubes, and centrifuged at 16,100 rcf for 10 minutes. The supernatants for 1 day passive release and treated for 2 hours were collected and assayed using the micro BCA protein detection kit.

Mass Spectrometry for Determination of Composition of Release Supernatant

To determine which proteins in hPM were released by composite microparticles after 2-hour exposure in an AMF, release supernatants were analyzed (in addition to basal hPM) by University of Florida Interdisciplinary Center for Biotechnology Research using Nano-LC-MS. For sample preparation, samples were centrifuged at 15000 g for 30 min at 4° C. The supernatant was collected and run into a NuPAGE Bis-Tris protein gel for 15 min according to the manufactural instruction (ThermoFisher Scientific). The gel was then stained with SimplyBlue Safe Stain (ThermoFisher Scientific) and de-stained with milli-Q water and further de-stained with the combination of acetonitrile and 50 mM ammonium bicarbonate (ABC) after slicing to small pieces. The protein samples in gel was then reduced by 45 mM dithiothreitol at 55° C. for 1 hour, followed by alkylation by 100 mM iodoacetamide in the dark for 45 min at room temperature. After that, all liquid was removed from the sample and the gel slices were washed with 50 mM ABC twice and dehydrated with acetonitrile and dried in SpeedVac™ (ThermoFisher Scientific). Trypsin (Promega, Fitchburg, Wis.), solubilized in 100 mM ABC (12.5 ng/μL) was added to cover the gel pieces for digestion overnight at 37° C. The digested peptides were desalted by ZipTip C18 resin (MilliporeSigma, MA) and dried in SpeedVac™ (ThermoFisher Scientific).

Peptides derived from the total proteins were suspended in 0.1% formic acid for mass spectrometric analysis. The bottom-up proteomics data acquisition was performed on an EASY-nLC 1200 ultraperformance liquid chromatography system (Thermo Scientific) connected to an Orbitrap Fusion Tribrid instrument equipped with a nano-electrospray source (Thermo Scientific). The peptide samples were loaded to a C18 trapping column (75 μm i.d.×2 cm, Acclaim PepMap® 100 particles with 3 μm size and 100 Å pores) and then eluted using a C18 analytical column (75 μm i.d.×25 cm, 2 μm particles with 100 Å pore size). The flow rate was set at 250 nL/minute with solvent A (0.1% formic acid in water) and solvent B (0.1% formic acid and 80% acetonitrile) as the mobile phases. Separation was conducted using the following gradient: 1-3% of B over 0-5 min; 3-30% of B over 5-95 min, 30-99% of B over 105-110 min, 1% of B over 115-120 min.

The full MS1 scan (m/z 350-2000) was performed on the Orbitrap with a resolution of 120,000 at m/z 200. The automatic gain control (AGC) target is 2e5 with 50 ms as the maximum injection time. Monoisotopic precursor selection (MIPS) was enforced to filter for peptides. Peptides bearing +2-6 charges were selected with an intensity threshold of 1e4. Dynamic exclusion of 30 s was used to prevent resampling the high abundance peptides. Top speed method was used for data dependent acquisition within a cycle of 3 s. Scan event one comprised an isolation window of 1.3 Da in the quadrupole mass analyzer, fragmentation of the selected peptides by collision induced dissociation (CID, 35% of normalized collision energy with 10 ms activation time). The MS2 spectra were detected in the linear ion trap with the AGC target as 1e4 and the maximum injection time as 35 ms.

The raw data were extracted to .mgf file using Proteome Discoverer v2.2.0.388 and employed to search against Uniprot Human protein database on Mascot 2.4. The search algorithm used 10 ppm and 1 Da as mass tolerance for MS1 and MS2, respectively. Trypsin, allowing a maximum of two missed cleavage sites, was selected for the enzyme digestion. Carbamidomethyl of cysteine (+57.021 Da) was set as the static modification, and oxidation of methionine (+15.995 Da) as well as deamidation on asparagine or glutamine (+0.984 Da) and peptide N-terminus, pyroglutamate (−17.026 Da) on glutamine were specified as the dynamic modifications. Scaffold 4 Proteome software was used to analyze the data using NCBI database with settings 95% Protein Threshold, 1 Minimum #of Peptides, 90% Peptide Threshold. These settings gave <0.5% decoy results.

Cell Culture

Cell culture experiments were performed with Warton's Jelly derived mesenchymal stem cells (MSCs) or human umbilial vein endothelial cells (HUVECs) between passages 3-6. MSCs were derived from Warton's Jelly (collected from UF Health Shands Hospital, Gainsville, Fla.) by separating Warton's Jelly from the umbilical cord and dicing it into small explants (<1 mm²). The explants were placed in petri dishes (35×10 mm, ThermoFisher Scientific) with culture medium (Dulbecco's Modified Eagle Medium (DMEM) with 10% fetal bovine serum (FBS) and 1% penicillin-streptomycin (penstrep)) and the MSCs diffused out of the tissue over a 3-week period. Endothelial cells were derived from human umbilical veins (collected from UF Health Shands Hospital, Gainesville, Fla.) after detachment from umbilical cord vessel walls using 1 mg/ml Type-1 collagenase as described in the literature²³⁷.

Endothelial cell media was used for culture of HUVECs (500 ml Vasculife Basal media, 25 ml glutamine, 0.5 ml hydrocortisone, 0.5 ml ascorbic acid, 10 ml FBS, 1.25 μl bFGF). MSCs were cultured with DMEM supplemented with 10% FBS and 1% penstrep until osteogenic experiments. For osteogenic experiments, MSCs were fed osteogenic media (445 ml DMEM, 50 ml FBS, 5 ml penstrep, 1.08 g β-glycerophosphate disodium salt hydrate, 19.6 μg dexamethasone, 0.029 g L-ascorbic acid 2-phosphate sesquimagnesium salt). All cells were cultured at standard conditions at 37° C. and 5% CO₂.

Calcein AM Staining of HUVECs

HUVECs were seeded at 2,048 cells/cm² in black 96 well-plates (Corning 3595) with a clear bottom. Cells were fed every other day with endothelial cell media (with or without hPM supplementation). On day 4, cells were washed with 200 μl PBS and stained with 5 mM calcein AM (Corning 35417) in media for 30 min at 37° C. Wells were then imaged with the Axio Observer Micoscope (Zeiss).

Alizarin Red S (ARS) Staining of Osteoblasts

MSCs were plated into 96 well plates (Corning 3595) at 2,048 cells/cm² and cultured for 7 or 14 days. MSCs were fed every other day with osteogenic treated media. For microparticle samples, 10-100 mg microparticles were suspended in 900 μl osteogenic media in sterile 1.5 ml centrifuge tubes. The samples were allowed to passively release for 24-hours and then were transferred to 8-well strips for 1-hour treatment (AMF 50 mT/224 kHz, 37°, or 72° C.). The sample was then transferred back to the 1.5 ml centrifuge tube under sterile conditions, and centrifuged for 10 min at 16,100 rcf. The supernatant was removed from the microparticle pellet and then filtered with sterile 0.45 μm PVDF filters to remove any excess particles. The supernatant (150 μL) composed of osteogenic media plus released hPM proteins was then added to the cells. As controls, osteogenic media was supplemented with 0.125% to 50% hPM.

On day 7 or 14, media was removed, and cells were washed twice with 80 μL PBS (without Ca²⁺ and Mg²⁺) before fixing with 80 μl 10% neutral buffered formalin for 1-hour. The wells were then washed with 160 μl distilled water. 160 μl 40 mM ARS (Sigma Aldrich A5533) in distilled water was added to each well and the plate was then covered with tin foil and incubated at room temperature on the shaker at 25 rpm for 45 min. The wells were then carefully washed with 160 μl distilled water until the solution came out clear (between 5-8 times). Distilled water was added one final time and the samples were imaged under a light microscope (Leica DFC295 or Zeiss Axio Observer).

After imaging, water was removed and the ARS was reacted with 225 μl 10% (w/w) cetylpyridium chloride (CPC, Sigma Aldrich C0732) overnight (12 hours) at 37° C. and then transferred to a 96-well plate with ARS/CPC standards. ARS/CPC standards were prepared from the 40 mM ARS stock solution with concentrations 0.0313 mM to 2 mM ARS. The plate was then read at 405 nm with pathlength correction using a spectrophotometer (Biotek Synergy 2). The amount of ARS was converted to the amount of calcium through a mole balance where 2 mole ARS=1 mole Ca²⁺.

Alamar Blue Cell Viability Assay

MSCs were plated in 96 well-plates at 6400 cells/well and cultured for 2 days before being assayed with alamar blue. Samples were fed with osteogenic media (treated or untreated) on day 1. Microparticle samples were treated the same as for ARS staining.

An alamar blue stock solution of 5 mM resazurin (Sigma Aldrich R7017) in distilled water was created and filtered with a 0.22 μm filter (Millipore SLGP033RS). Media was removed and each well was washed with PBS (without Ca²⁺ and Mg²⁺). For cell viability, a solution of 10% alamar blue stock+media (DMEM+10% FBS+1% PS) was prepared and 110 μl was added per well. For controls, wells with no cells and with untreated cells were used. Cells were incubated with the alamar blue solution for 2 hours at 37° C. and then solutions were transferred to a black 96 well-plate with a clear bottom. The fluorescence signal was read using a spectrophotometer (Biotek Synergy 2) at 540-570 nm excitation, 580-610 nm emission.

Data Analysis

For statistical analysis to determine if triggered release in an AMF or at 72° C. was significantly different than passive release at 37° C., a non-paired two-tailed t-test was used (α=0.05). Data were plotted using Microsoft Excel and SigmaPlot.

### Results and Discussion

Effect of Double Emulsion Parameters on Composite Microparticle Properties

Composite microparticles were synthesized via a double emulsion as described in Section 4.2.3. Many parameters in the double emulsion were varied including molecular weight of the PCL, the solvent evaporation method/time, homogenization times, and the emulsifying agent for W2 phase. For the solvent evaporation, magnetic stirring was compared to an orbital shaker plate. Magnetic stirring resulted in less magnetic loading, with many MNPs being stuck to the magnetic stir bar, and “crushing” some of the microparticles. Conversely, the orbital shaker resulted in spherical particles with 78% MNP loading efficiency.

The speed and method of mixing the primary emulsion was varied to manipulate the size of the microparticles, as the mixing speed during this step provides energy to disperse the oil throughout the water phase. It was found that increasing the speed of the primary emulsion resulted in smaller hydrodynamic diameters. When the primary emulsion time was increased from 20 sec to 2 min, the average microparticle size decreased from ˜50 μm to ˜30 μm, and protein loading increased from ˜20% to ˜30%. In addition, sonication was tried for the primary emulsion rather than homogenization, and this resulted in 0% protein loading. When the time of the primary emulsion was further increased to 4 min homogenization, the particle size decreased to ˜15 μm.

Different emulsifying agents were tested for use in the W2 phase including 0.5% (w/v) sucrose, 0.5% (w/v) polyethylene glycol (PEG), and 0.5% (w/v) PVA. The concentration, 0.5% (w/v), was used for all emulsifying agents as this concentration (and lower) was found to not interfere with the micro BCA protein detection assay. Sucrose and PEG emulsifiers were unable to form spherical particles when no protein was added to the W1 phase for formulation of vehicle controls, and rather “clumps” were formed. This indicates that the protein was stabilizing the emulsion rather than the emulsifying agent, and this led to lower protein loading within the microparticles. 0.5% PVA was able to form stable spherical particles even without protein in the W1 phase, and thus was chosen as the emulsifying agent for further syntheses.

Composite microparticles were also synthesized using various molecular weights of PCL (1,040 Da, 14,000 Da, and 80,000 Da) and analyzed by DSC for melting temperatures shown in Table 1.1. DSC was ran for two cycles, and after microparticles melted and re-solidified, their melting temperatures decreased, likely due to a change in crystallinity. This will be useful to keep in mind when considering multiple cycles of release. The protein loading for all groups was within 10% of each other. However, according to preliminary release studies, proteins were released almost immediately from the 1,040 Da PCL microparticles, indicating the protein was not encapsulated but more likely stuck to the polymer and thus all released once exposed to water. Thus, 14 kDa PCL was used as it had the next lowest melting temperature.

TABLE 1.1
Melting temperatures for composite microparticles made
of different molecular weight PCL as determined by DSC.

| Molecular Weight | Tₘ₁ (° C.) | Tₘ₂ (° C.) |
|---|---|---|
| Mₙ~1,040 | 54.61 | 41.83 |
| M_{w}~14,000 | 63.31 | 53.83 |
| Mₙ~80,000 | 64.46 | 55.86 |

Previous studies have also sought to evaluate the effect of double emulsion synthesis parameters on properties of PCL microparticles. A study by Yang et al.²³⁸ synthesized PCL microparticles and found that stirring speed was the dominating factor for microparticle size with higher stirring speeds resulting in smaller microparticles; increasing the PVA percentage (0.05% to 0.5%) in the W2 phase resulted in a decrease in microparticle size and a reduced release rate of bovine serum albumin (BSA) due to increased stability of the emulsion; low volume of the O phase and higher polymer concentration in the O phase resulted in larger microparticles with more pores; and lower BSA loading led to lower burst releases due to decreased concentration gradient with the outer water phase. Another study by Lamprecht et al.²³⁹ synthesized microparticles made from 50/50 PLGA/PCL and found that increasing polymer concentration increased size and polydispersity; increasing the homogenization time increased particle size and polydispersity (which is the opposite of what was found in the current study); increasing PVA percentage decreased the size of the microparticles; and increasing the amount of BSA in the W1 phase decreased the encapsulation efficiency. Lasty, a study by Benoit et al.²⁴⁰ synthesized PCL microparticles and found similar findings to the current study and the other previous studies; first, it was found that increasing polymer concentration in the O phase increased microparticle size and resulted in maximum protein loading at 5% polymer concentration and 4.33±0.52 μm microparticle size; increasing PVA in the W2 phase decreased microparticle size; and increasing mixing time for the emulsions decreased microparticle size.

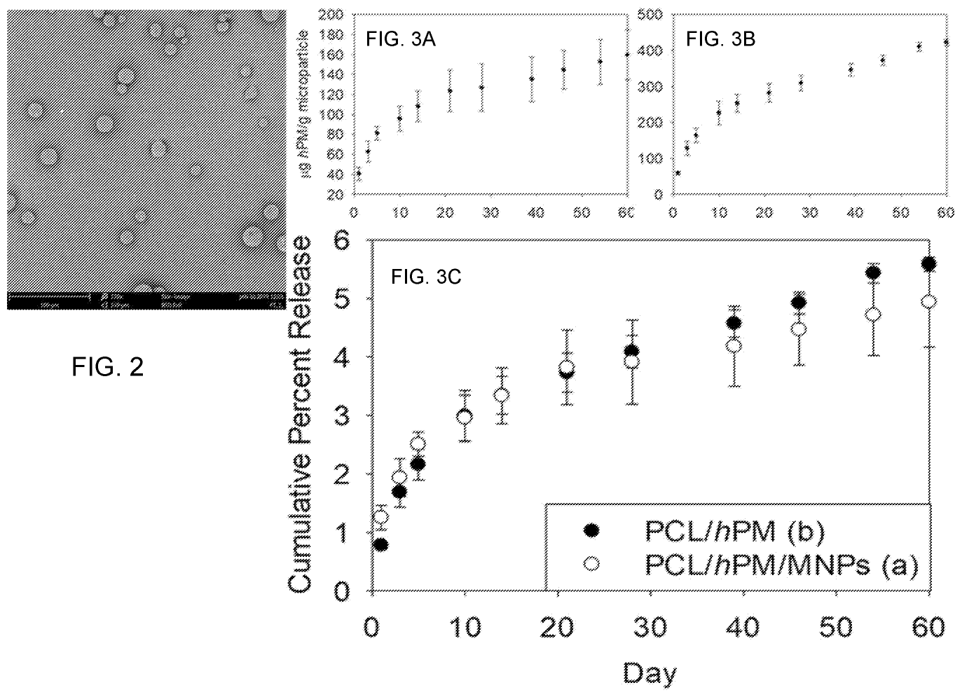

Final synthesis parameters in the current study with M_{w} 14,000 PCL resulted in composite microparticles with a hydrodynamic diameter of 15.69±7.01 nm and protein loading percentage of 30.4%. The MNP loading percentage was 78%. An SEM of composite microparticles can be seen in FIG. 2.

Loading Method Accuracy

The hydrolysis method for loading was found to give the most accurate results for protein loading. The dissolution method resulted in high error with over 100% standard deviations. It also had a film form between the aqueous and organic phases, and it is possible protein got trapped within this film leading to the high errors. The indirect method often gave unreasonably high loading percentages (over 100% when PVA controls were taken into account) due to the loss of proteins on the synthesis equipment (e.g. homogenizer and beakers).

Passive Release for 60 Days

hPM release was measured for up to 60 days from composite microparticles in addition to PCL microparticle with hPM only (no MNPs). Release is shown in FIGS. 3A and 3B as μg protein/g microparticle. The release was corrected for by subtracting the release signal of PCL only particles from the PCL/hPM sample and subtracting the release signal of PCL+MNPs from the PCL/hPM/MNPs sample. As noted below, protein loading into composite microparticles was lower than the loading into PCL only microparticles without MNPs, likely due to the MNPs taking up space within the composite microparticles. Thus, after adjusting for mean loading, the release was approximately the same (around 5%) as shown in FIG. 3C. While this is a low value for the percent protein release, PCL is known for its slow-release profiles and has been shown in the literature to release low protein amounts: <30% total release of BSA for 28 days from PCL microparticles (23.66±0.12 μm; 5% PVA emulsifier; 96.1 kDa PCL) (see e.g., Youan, B. B. C., Benoit, M. & Baras, B. Protein-loaded PCL microparticles. Optimization of the preparation by (water-in-oil)-in water emulsion solvent evaporation. J. Microencapsul. 16, 587-599 (1999)); <10% release of BSA after 40 days from PCL microparticles (97.6 μm; 80 kDa PCL) (see, e.g., Yang, Y. Y., Chung, T. S. & Ping Ng, N. Morphology, drug distribution, and in vitro release profiles of biodegradable polymeric microspheres containing protein fabricated by double-emulsion solvent extraction/evaporation method. Biomaterials 22, 231-241 (2001)); <4% release of BSA after 40 days from PCL wet extruded scaffolds (80 kDa PCL; 180 mg tubular scaffold/5 mm long) (see, e.g., Ozkan, S., Kalyon, D. M., Yu, X., McKelvey, C. A. & Lowinger, M. Multifunctional protein-encapsulated polycaprolactone scaffolds: Fabrication and in vitro assessment for tissue engineering. Biomaterials 30, 4336-4347 (2009)).

In addition, in the current study the release was double-checked by hydrolyzing the remaining particles and measuring the amount of protein still encapsulated after the 60 day release timepoint. Further, hPM can induce osteogenesis even at very low amounts, so the low release amount will not hinder the effectiveness of the release system. In fact, the relatively low burst release could be advantageous as it will allow for protein release over longer time periods, which may be required for nonunion fractures which take up to 9 months or for proper healing.

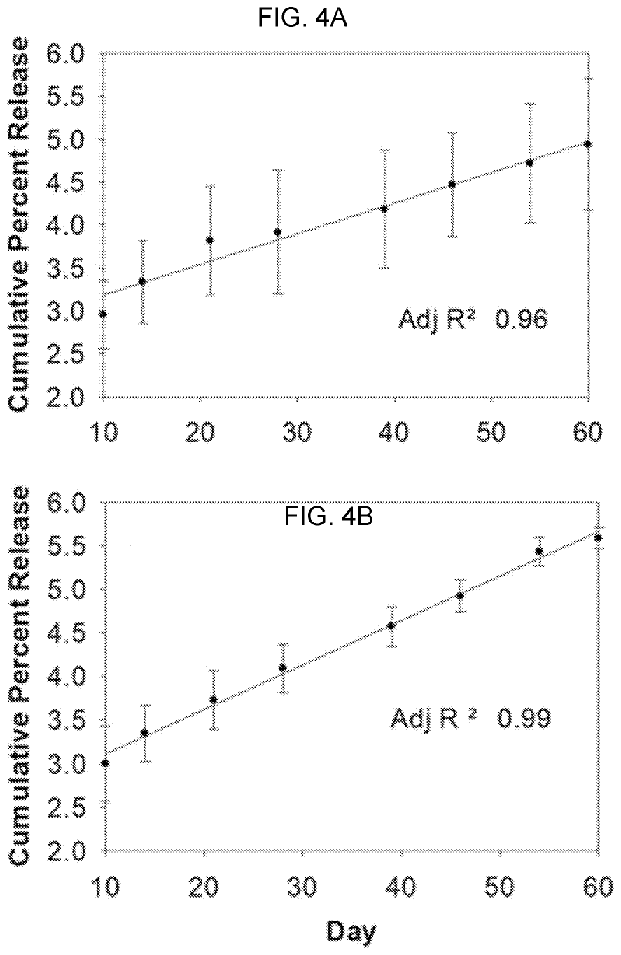

The release appears to be a burst release followed by zero-order passive release. The data were fit to linear fits for day 10 and beyond (see FIG. 4) and it was found that the adjusted R² values were 0.96 for composite microparticles with MNPs and 0.99 for PCL microparticles (no MNPs). As multiple proteins are being released from the system, it is difficult to distinguish which ones are being released at which time points, and they may follow different transport mechanisms based on their respective sizes.

Triggered Release

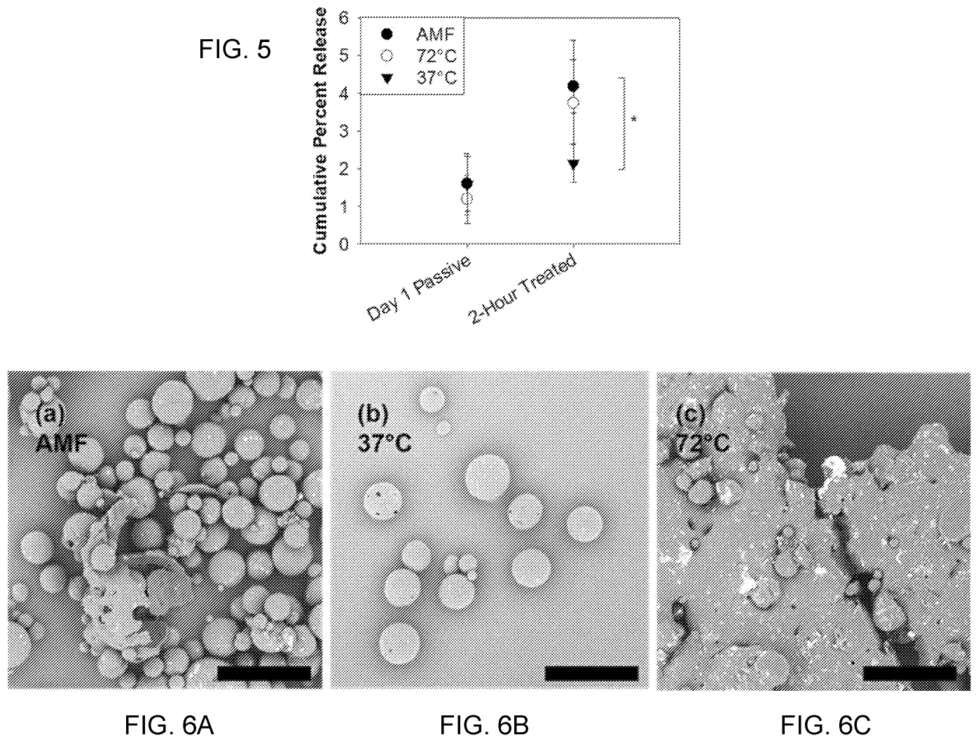

For triggered release experiments, composite microparticles were allowed to passively release for 24-hours to account for the burst release and to show that all samples had the same basal release levels. Then, resuspended in distilled water and exposed to different conditions for 2-hours: AMF, 72° C. as a positive heat control, and 37° C. as a passive release control. As shown in FIG. 5, AMF triggered release was significantly higher than passive release at 37° C. Samples exposed to 72° C. released higher amounts of hPM than samples maintained at 37° C., but it was not significant, most likely due to the high standard deviation for the 72° C. release. The release after 2 hour treatment in an AMF or at 72° C. was comparable to passive release achieved after 30 days in FIG. 4-5. Samples reached a bulk temperature of ˜55° C. in the AMF.

SEM images (see FIGS. 6A-6C) were taken of each sample after treatment, and the AMF and 72° C. samples displayed signs of polymer melting whereas the particles in the sample maintained at 37° C. remained spherical. As shown in FIG. 6A, not all particles exposed to the AMF displayed signs of melting, indicating that the MNP distribution throughout the MNPs may not have been uniform, with microparticles encapsulating more MNPs melting more rapidly in the applied AMF.

Characterization of hPM and Released Proteins

The protein concentration of hPM was found to be 5.8±2.3 mg/ml using the Pierce BCA protein detection kit. The proteins in hPM and in the supernatant from release experiments under AMF exposure (i.e. hPM that was released from composite microparticles) were evaluated using mass spectrometry. As shown in Table 1.2, hPM includes proteins involved in many facets of bone regeneration including inflammatory, angiogenic, osteogenic, ECM, remodeling and clotting proteins. Thus, it is reasonable that hPM will be able to induce regeneration. As shown, over 80% of the proteins in the basal hPM were successfully encapsulated within microparticles and released after 2 hours of exposure to an AMF. As discussed in below, the released proteins were capable of inducing Ca²⁺ deposition in differentiated osteoblasts at levels comparable to basal hPM.

TABLE 1.2

| | | | Released |
|---|---|---|---|
| Content | | hPM | hPM |
| Total Number of Proteins | | 1164 | 967 |
| Inflammatory | Complement C3 | + | + |
| | Complement C5 | + | − |
| | Complement C9 | + | + |
| | Interleukin Enhancer Binding | + | + |
| | Factor 3 | | |
| | Interleukin-27β | + | + |
| | Prostaglandin | + | − |
| | Annexin | + | + |
| Angiogenic | TGF-β | + | + |
| | Profilin-1 | + | + |
| | Myeloid-Derived Growth Factor | + | + |
| | VEGF | + | − |
| | PDGF | + | − |
| | Ribonuclease/Angiogenin Inhibitor 1 | + | + |
| | Thrombospondin 1 | + | + |
| | Angiotensinogen | + | + |
| Osteogenic | Growth Hormone B1 | + | + |
| | Osteoglycin | + | + |
| | Periostin | + | + |
| ECM | Fibronectin | + | + |
| | Laminin | + | + |
| | Collagen | + | + |
| | Nidogen 1 | + | + |
| | Lumican | + | + |
| | Vitronectin | + | + |
| | Prolargin | + | − |
| Remodeling | Growth Factor Receptor Bound | + | − |
| | Protein 2 | | |
| | Heat Shock Protein | + | + |
| Clotting | Coagulation Factor XIII | + | + |
| | Coagulation Factor IX | + | + |
| | von Willebrand factor | + | − |
| | Fibrinogen | + | + |

In Vitro Optimization of hPM Culture Conditions

In previous studies, hPM was coated onto the bottom of the tissue culture plate and other culture conditions were not investigated³⁰. In this study, further culture conditions of hPM were investigated to improve its in vitro effects. Cell culture media was supplemented with hPM from concentrations 0.125% to 50% hPM and compared to the hPM coating.

First, the effect of hPM in the media was evaluated in HUVECs, and the coating did not induce vascular network formation as it did in previous studies (results not shown). It is possible in previous studies the coating was broken up due to pipetting and was actually suspended in the media, more like 25% hPM. In the current study, pipetting was performed very carefully in order to ensure that the coating was not broken off the bottom of the plate. Samples fed with 5 to 25% hPM showed vascular network formation. Samples fed 50% hPM did not show vascular network formation and it is possible that this is because this concentration of hPM was too dense for healthy cells to grow.

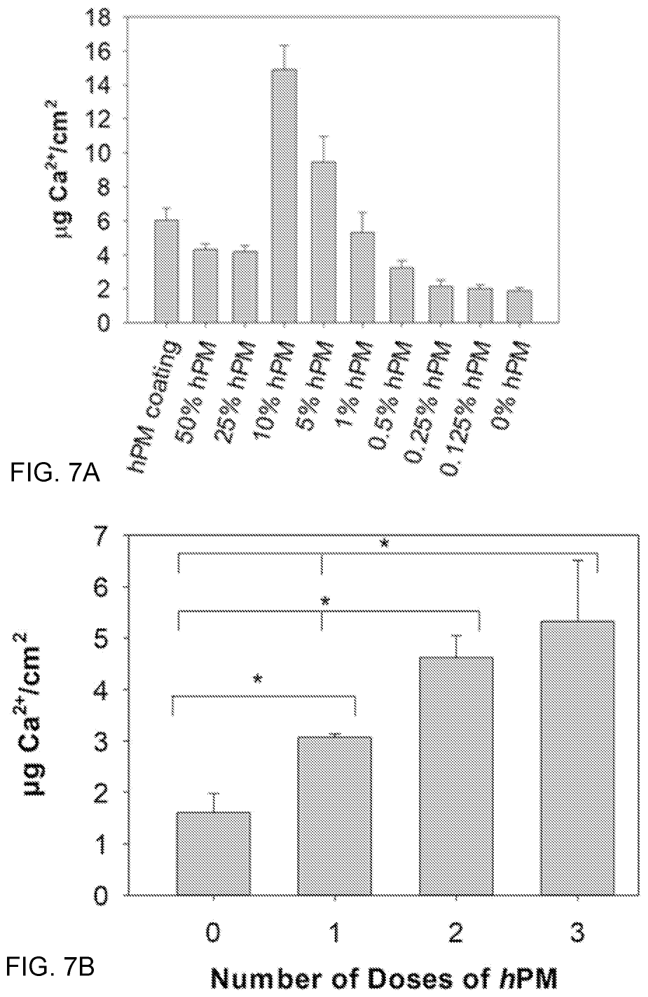

Next, hPM was added as a supplement to osteogenic media and cultured with MSCs, and the results are shown in FIG. 4-9. The first experiment analyzed concentrations 5% hPM to 50% hPM and were imaged (not shown) on the Axio Observer (Ziess). The second experiment analyzed 0.125% hPM to 1% hPM and were imaged (not shown) on the Leica DFC 295. To summarize the results, the samples were reacted with CPC and the absorbance was measured and quantified as illustrated in FIG. 7. As shown, 10% hPM in the media induced the highest amount of Ca2+ deposition.

In addition, experiments were done with MSCs cultured for 7 days and fed every other day with osteogenic media, with or without supplemented 1% hPM, as described in the methods. The sample fed 3 doses of hPM were fed with hPM supplemented media every time, the sample with 2 doses were fed with hPM supplemented media on day 1 and 3 and fed with osteogenic media on day 5, the sample with 1 dose was fed with hPM supplemented osteogenic media on day 1 and fed with osteogenic media on days 3 and 5, and the sample with 0 doses was only fed osteogenic media. The sample fed 3 doses of hPM had the highest Ca²⁺ deposition at the end of the week, and the amount of Ca²⁺ deposition was shown to positively correlate with the amount of times the cells were fed with hPM supplemented osteogenic media versus plain osteogenic media as shown in FIG. 7B. This data illustrates that a magnetically triggered release platform capable of triggering multiple doses of hPM will be useful for induction of increased osteogenesis.

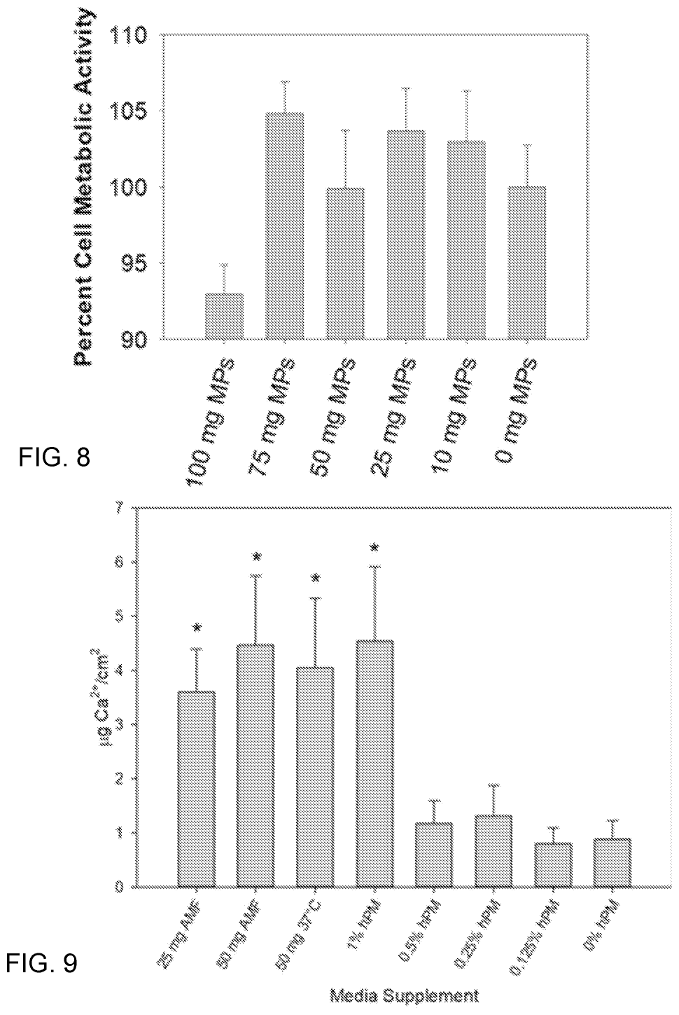

For triggered release experiments with microparticles, the particles were suspended in osteogenic media and allowed to release the hPM into the media under passive release conditions at 37° C. or under exposure to an AMF. The particles were then centrifuged and filtered from the media, and the media with released hPM were added to the cells. A toxicity study was performed with the microparticles at different concentrations in osteogenic media exposed to an AMF and showed that doses 75 mg/900 μL and below were nontoxic by alamar blue assay (FIG. 8). However, cell morphology appeared different in samples above 25 mg/900 μL, suggesting the cells could be unhealthy. Therefore, 25 and 50 mg samples were tested in the in vitro experiment described below.

In Vitro Evaluation of the Activity of Protein Released from Triggered Release Platform in Osteoblasts

MSCs were cultured with osteogenic media that was supplemented with hPM at different percentages or hPM released from composite microparticles exposed to an AMF or by passive release at 37° C. as described in the methods, above. Differentiated osteoblasts were stained with ARS on day 14. hPM released into osteogenic media by composite microparticles was estimated to be between 0.5%-1% hPM. As shown in FIG. 9, calcium deposition as determined by ARS staining was significantly higher than the osteogenic media control for both 25 mg and 50 mg samples exposed to an AMF as well as 50 mg at 37° C. The 50 mg exposed to AMF gave a slightly higher average calcium deposition value than 50 mg maintained at 37° C. Additionally, all of the composite microparticles gave average release values that were between 0.5% hPM-1% hPM, although the values were closer to 1% hPM. In addition, the ARS stain was darker red in composite microparticle samples compared to the osteogenic media control (not shown). These results indicate that the released hPM maintains its functionality. In future experiments, control samples with no loaded hPM will be evaluated.

### Conclusions

Composite PCL microparticles with dispersed MNPs and loaded hPM, a model protein mixture, have been synthesized and characterized. The composite microparticles were shown to sustain passive release of hPM for at least 60 days and trigger hPM release under an applied AMF at a defined time point. The system holds promise as a method to deliver a variety of growth factors and other large biomacromolecules on demand while retaining the functionality of the proteins. In fact, this is the first developed magnetically triggered release platform for a protein mixture, and thus provides insight into development of triggered release systems for future studies. The developed platform has the potential to enhance cell activity within engineered scaffolds for bone healing and other tissue engineering applications. In addition to the release platform, culture conditions for the human placental proteins were evaluated in two cell types.

### Example 2—the Effect of Synthesis Parameters on Properties of Composite Microparticles (Hydrodynamic Diameter, Dispersity, Loading Efficiencies, Crystallinity, and Release Kinetics)

PCL microparticles with encapsulated hPM and SPIONs were used in the example above. In addition to the features discussed above, it is desired that embodiments of the composite microparticles of the present disclosure not passively release all of the encapsulated hPM (or other payload) before magnetic triggering is employed. Microparticles prepared via a double emulsion can be used for hydrophilic or hydrophobic payloads, and parameters of the double emulsion have been shown to affect the properties of the product microparticles⁵⁸. The diameter, dispersity, loading efficiencies, and crystallinity of the microparticles can affect the release kinetics of the hPM, and will therefore be evaluated in regards to varying double emulsion parameters.

PCL has been used for long term protein delivery, such as bovine serum albumin (BSA)²⁷, nerve growth factor (NGF)²⁸, and insulin⁵⁹. However, it is not believed that PCL microparticles have been prepared or used for release of a heterogeneous protein mixture such as hPM. Additionally, double emulsion parameters for PCL have been previously evaluated.⁵⁸ In the present example, release kinetics can be optimized by varying microparticle properties through changes in the double emulsion synthesis. In the present example, instead of hPM, model protein, BSA, was used for evaluation of microparticle properties based on formation parameters.

### Methods:

PCL microparticles were synthesized via water/oil/water double emulsion (W1/O/W2) followed by lyophilization. Briefly, PCL of different molecular weights (Mₙ˜1200 Da, M_{w}˜14,000 Da, Mₙ˜80,000 Da) were dissolved in dichloromethane (O), and a model protein, BSA, was added to aqueous tris buffer at mg/ml concentrations (W1) (as hPM is stored in tris buffer and has a comparable concentration). W1 was added to O and homogenized. Then, W1/O was added to W2, which included poly(vinyl alcohol) (PVA) dissolved in water, and was homogenized. W1/O/W2 was stirred overnight using a shaker plate at 175 rpm to allow for solvent evaporation. Then, microparticles were centrifuged, washed with distilled water three times, and lyophilized. The dry microparticles can be stored at 4° C. Double emulsion parameters such as amount of polymer used, O to W1 volume ratio, W1/O to W2 volume ratio, protein concentration in W1, PVA concentration in W2, molecular weight of polymer, primary and secondary emulsion homogenization times, evaporation time, number of washes, and mechanism of stirring for evaporation were in order to evaluate synthesis parameters.

Characterization of product microparticles: Microscopy was used to evaluate average hydrodynamic diameter and dispersity. ImageJ software was used to analyze microscopy images. Loading efficiencies were evaluated by quantifying remaining protein in supernatant after washes using a BCA Protein Assay. Yield can be calculated by weighing the product microparticles after lyophilization. Release experiments were performed by submersing 10 mg of microparticles in 1 ml of phosphate buffered saline (PBS) and mixing on a shaker plate at 37° C. Microparticles were centrifuged and, PBS supernatant was removed and assayed using BCA Protein Assay. New PBS was added after each measurement. Sodium dodecyl sulfate polyacrylamide gel electrophoresis (SDS-PAGE) was used to determine if the BSA was fragmented after release. Differential scanning calorimetry was used to determine the melting point and crystallinity of microparticles.

After microparticles that are able to sustain release of BSA over time were synthesized, SPIONs can be added into the O phase during the double emulsion. As all SPIONs evaluated in Example 1, above, were hydrophilic, and as such were functionalized with oleic acid. Release of BSA will be characterized as described to see how SPION incorporation affects the release kinetics. In addition, loading efficiency of magnetic particles will be evaluated via an ultraviolet-visible (UV-Vis) colorimetric technique using o-phenanthroline complexes.

As described above in Example 1, Microparticles were also synthesized with hPM instead of BSA as and characterized as described above.

### Results:

Many double emulsions have been run to try and obtain particles within specified size ranges determined from preliminary calculations, to reduce dispersity, and to maximize loading. In the beginning, there were troubles with particle aggregation due to wash steps and amount of PVA stabilizer in W2. There was also an issue that microparticles were being “crushed” by the magnetic stir bar during the overnight solvent evaporation. As seen in Table 2.1, these two issues have been resolved and particles that are approximately the desired size have been obtained. The procedure for microparticles in the desired size range includes a 4 minute homogenization (variac 20) and a 2 minute homogenization (variac 40) for the secondary emulsion. The variac is a voltage transformer that has been used in order to alter the speed of the homogenizer, which is normally 20,000 rpm. This procedure was done for all three molecular weights of PCL, and there was not a large change in properties of microparticles with regards to size, loading, and dispersity. When the Mₙ˜80,000 PoL was used, a slight decrease in loading was observed, which was unexpected.

The centrifugation speed during washes will be altered to try and increase the yield, which right now is between 25-50%. In addition, to increase loading above 30%, the volume of W1 or concentration of protein in W1 will be altered. A concentration comparable to hPM has been used previously, but the hPM could be diluted if this will increase loading efficiency.

TABLE 2.1
Table of parameters in the double emulsion that have been changed
and the key result this had on microparticle properties.

| Double Emulsion Parameter Changed | Key Result on Microparticle Properties |
|---|---|
| Altered W2 volume, 25 or 50 ml | No significant difference |
| Altered amount of washes, 1 to 3 | 3 washes sometimes results in particle |
| | aggregation, need to take image on |
| | microscope after every wash to make sure |
| | aggregation does not occur |
| Altered PVA % in W2, 0.5% to 5% | 5% most stable particles, least amount of |
| | aggregation |
| Altered evaporation stirring technique, | Magnetic stirring results in “crushing” of some |
| magnetic stirring or shaker plate | particles but shaker plate does not |
| Altered size of magnetic stir bar used for | Smaller magnetic stir bar results in less |
| magnetic stirring | “crushed” particles |
| Increased primary emulsion homogenization | Average particle size decreased from 50 μm |
| time, 20 sec to 2 min | to 30 μm, loading increased |
| | from ~20% to ~30%, |
| Sonicated primary emulsion instead of | 0% loading after sonication |
| homogenized | |
| Increased speed of secondary emulsion | Particle size decreased from to 20 μm, |
| homogenization, variac voltage transformer | dispersity decreased |
| set to 20 or 40 | |
| Increased secondary emulsion | Particle size decreased to ~10 μm |
| homogenization time, 1 min to 2 min (keeping | |
| high speed) | |
| Increased primary emulsion homogenization | Particle size decreased to less than 5 μm, no |
| time, 2 min to 4 min (keeping high speed | effect on loading |
| longer secondary emulsion) | |

In addition to this, DSC data for microparticles of different molecular weights was also tested to see how the molecular weight affects the melting temperature of the particles. These results can be seen in Table 1.1 above. DSC was run for two cycles, and after the microparticles melted and re-solidified, their melting temperatures decreased, likely due to a change in crystallinity. This will be useful to keep in mind when considering multiple cycles of release.

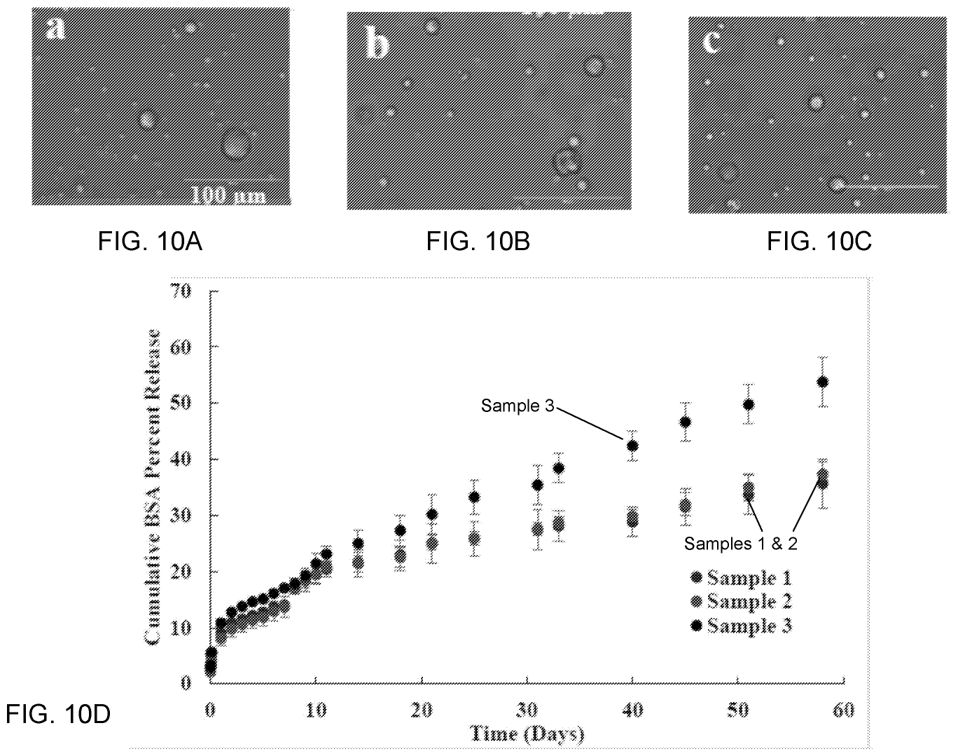

PCL microspheres encapsulating BSA were made as described above and shown in FIGS. 10A-10C. Passive release of BSA from microparticles was also tested. As seen in FIG. 10D, PCL microparticles (M_{w}˜14,000) successfully released BSA for 58 days. This shows that magnetic triggering is a possibility, as not all of the BSA is released passively in this time period. Release for these particles under various conditions, such as temperature changes, will also be evaluated

If use of dichloromethane denatures certain proteins, use of nontoxic solvents can be used in the double emulsion, such as anisole, which has been identified to work in the synthesis of PCL microspheres⁶⁰. Alternative synthesis techniques such as spray drying³² or electrospinning⁶¹, can also be evaluated. In addition, geometry could be changed from spherical to a thin film or cylinder, which will affect release kinetics. Polymer blends of PCL:poly(lactic-co-glycolic acid) or other polymers with low melting temperatures, such as polyvalerolactone, could also be evaluated.

### Example 3—Evaluate Heat Generation in an AMF for Commercially Available SPIONs with Different Surface Chemistries, Hydrodynamic Diameters, and Magnetic Core Diameters

In other examples of the present disclosure, SPIONs are used for their ability to generate heat in an applied AMF in order to facilitate PCL melting and enhanced hPM diffusion. Commercially available MNPs are frequently used for in variety of research applications, and thus it would be useful for researchers to know their properties, particularly their magnetic characteristics. Unfortunately, commercially available MNPs are often not fully characterized or characterization data is limited. The SAR values for MNPs remain unreported in many cases. Thus the present Example analyzes this heat generation for various SPIONs.

For this Example, the heating potential of several commercial MNPs was measured in two different magnetic fields and quantified based on calculated SAR values and intrinsic loss parameters (ILP). Additionally, MNP properties including iron content, hydrodynamic diameter, magnetic diameter, core diameter, magnetically dead layer thickness, and saturation mass magnetization were evaluated. Correlations between these parameters and SAR values were analyzed using Pearson's product moment and Spearman's Rank correlation coefficients. Identification of any specific properties of the MNPs which result in high SAR values will have a pivotal impact on the development of the next generation of MNPs for hyperthermia. To standardize the SAR values, SAR was calculated as energy per MNP mass and as energy per iron mass, as both of these annotations are commonly found in the literature. For SAR calculation, the initial slope method (ISM), corrected slope method (CSM), and Box-Lucas method (BLM) were used and validated. Evaluation of the SAR calculation methods and units was performed to give researchers an idea of the variation of results that can be obtained due to different data analysis methods, and highlight the importance of specifying calculation method and units. The present Example provides an overview and discussion of the detailed analysis of commercially available SPIONs conducted in our lab and described in Lanier O L, Korotych O I, Monsalve A G, Wable D, Savliwala S, Grooms N W F, Nacea C, Tuitt O R, Dobson J. (2019). Evaluation of Magnetic Nanoparticles for Magnetic Fluid Hyperthermia. Int J of Hyperthermia, 36(1): 687-701, which is incorporated herein by reference. A general summery of the methods, results and discussion is provided below.

Commercially available SPIONs were evaluated rather than SPIONs synthesized in lab for both convenience and quality control. Commercially available SPIONs are produced with monodisperse populations and high reproducibility. SPIONs with a high SAR/ILP will be valuable to many researchers who want to study magnetically triggered release or magnetic fluid hyperthermia. Magnetic fluid hyperthermia is currently being employed in clinical trials for cancer treatment around the world, and has been shown to improve the efficacy of chemotherapy and/or radiation treatment³⁹. One of the pitfalls of hyperthermia treatment is the inability to deliver sufficient heat to the tumor due to insufficient amounts of SPIONs in the tumor, unequal distribution of SPIONs throughout the tumor, or loss of heat due to blood flow^{48,56}. Therefore, identification of high SAR/ILP commercially available SPIONs could also have a pivotal impact to the development of hyperthermia treatment for cancer. Outside of the biomedical field, SPIONs with high SAR/ILP could be used as catalysts for reactions in microfluidic reactors⁵⁷.

Finally, this work will evaluate if ILP is able to appropriately correct the SAR for field strength and frequency. If a high amount of variation in ILP is seen between samples measured at different field strengths and frequencies, a new parameter will be empirically derived.

Two previous studies have sought to evaluate the heating potential of commercially available SPIONs^{47,55}, but the variety and number of the SPIONs evaluated were limited. This example builds on the previous work to include more companies and products. Since SAR/ILP values have high variation between labs due to poor standardization of measurement conditions⁴⁸, this study analyzed a wide variety of particles in one lab setting to provide an accurate direct comparison.

SAR/ILP values of commercially available SPIONs can be influenced by several factors, such as on amount of iron in the sample, magnetic core diameter, surface chemistry, hydrodynamic diameter, magnetic field strength and frequency, and particle concentration. Materials and methods are described in full below, but results and discussion are summarized.

### Materials & Methods:

Materials. Hydroxylamine hydrochloride (PN 431362, 99.999% trace metals basis, ACS Reagent Grade, Sigma-Aldrich), nitric acid for ultra-trace elemental analysis (PN A467-2, 67-69%, Optima™, Fisher Chemical, Fisher Scientific), anhydrous sodium acetate (PN S8750, more than 99.0%, ReagentPlus®, ACS Reagent Grade, Sigma-Aldrich), 1,10-phenanthroline monohydrate (PN 77500, more than 99.0%, ACS Reagent Grade, Sigma-Aldrich), and iron standard for ICP (PN 56209, 10,000 mg/L Fe in nitric acid, certified reference material, TraceCERT®, Sigma-Aldrich) were used for iron quantification.

Thirty one commercial magnetic nanoparticles were tested: fluidMAG/C11-D (Chemicell), carboxylic acid functionalized iron oxide nanoparticles (Imagion Biosystems), mPEG coated iron oxide nanoparticles (Imagion Biosystems), 09-56-132 (Micromod), 09-56-252 (Micromod), 102-00-132 (Micromod), 104-00-501 (Micromod), 79-01-102 (Micromod), SEI-10-05 (Ocean NanoTech), SHA-10-02 (Ocean NanoTech), SPP-05-02 (Ocean NanoTech), HyperMAG A (NanoTherics), HyperMAG B (NanoTherics), HyperMAG C (NanoTherics), 544884 (Sigma-Aldrich), 637149 (Sigma-Aldrich), 725331 (Sigma-Aldrich), 747254 (Sigma-Aldrich), 747335 lots MKBW8617V and MKCD1194 (Sigma-Aldrich), 747440 (Sigma-Aldrich), 747459 (Sigma-Aldrich), 747467 (Sigma-Aldrich), 773352 (Sigma-Aldrich), 900026 (Sigma-Aldrich), 900027 (Sigma-Aldrich), 900028 (Sigma-Aldrich), 900042 (Sigma-Aldrich), 900043 (Sigma-Aldrich), 900062 (Sigma-Aldrich), and 900146 (Sigma-Aldrich).

SAR measurements. AMFs were applied to samples using an Easyheat™ induction heater (Ambrell). The experimental setup is shown in Figure S1. Two different types of water-cooled (set-point 18° C.) copper solenoid coils were utilized to test the heating potential of MNPs at two different field strengths and frequencies. The first coil (vertical; length=0.072 m; diameter=0.032 m; n-turns=8) operates at 401.1 A and 345 kHz, with a measured field strength of 74 mT, while the second (horizontal; length=0.135 m; diameter=0.045 m; n-turns=14) operates at 270.9 A and 223 kHz, with a measured field strength of 51 mT. Field strengths Bₐ (T) were calculated according to the formula:

$${B}_{a} = \frac{{U}_{a}}{{S}_{a} \cdot {f}^{′}} \qquad ( 1 )$$

where Uₐ is the root mean square voltage measured using an AMF probe (Life Systems) (V);

Sₐ is axial sensitivity factor of the probe;

ƒ is the frequency (kHz).

Aqueous dispersions of MNPs (200 μL, 1 mg MNP/mL) were measured in the center of the coil at least in triplicate. MNP concentration was expressed in mg MNP/mL rather than in mg Fe/mL due to two reasons: (1) most suppliers provide MNP dispersion concentration as mg MNP/mL, and (2) MNP samples with low iron content will have high nanoparticle concentration which can potentially cause sample aggregation. Custom 3-D printed polymer holders were used to precisely place the samples in the center of the solenoid. Distilled water was used as a control sample for both fields. Fiber optic temperature sensors (NeOptix; OD=1 mm) were used to measure the sample and coil temperature for 180 s total in 1 s intervals: 30 s with no field application, 120 s with field application, followed by 30 s of no field application. Pyrex vials (ID=6.0 mm; OD=8.0 mm; height=45.0 mm, V=1.24 mL) were used for the 74 mT coil, while polystyrene 8 stripwell (PN 9102, Corning; ID=6.4 mm; OD=8.5 mm; height=12.0 mm, V=0.36 mL)—for the 51 mT coil. Prior to the next measurement the coil was cooled down to 18° C.

The coil system was set-up within a styrofoam insulted box (Figure S1) to decrease temperature fluctuation. For the coil operating at 345 kHz/74 mT, styrofoam was also placed around the sample holder to ensure further insulation of the sample, while for the 223 kHz/51 mT coil the 3-D printed plastic holder for the sample provide sample placement in the middle of the coil and its insulation. Due to the different set-ups, heat loss may differ slightly at the two field strengths; however, all samples were treated the same way at each field strength for internal consistency. Parafilm® was used to seal the vials with MNPs sample. Three temperature sensors were inserted through the parafilm and positioned in the middle of each sample, while the forth sensor was used to record the coil temperature.

SAR calculations were performed using the ISM, the CSM, and the BLM. The formula for SAR calculation using the initial slpe method is [34]:

$${SAR}_{ISM} = \frac{Δ T}{Δ t} \cdot \frac{C ρ}{{m}_{F e / M N P s}} , \qquad ( 2 )$$

where ΔT/Δt is the slope of the first 60 s of AMF exposure (K/s);

C is the heat capacity of fluid (J/(K·g));

ρ is the density of the fluid (g/mL);

m_{Fe/MNPs} is the concentration of iron or MNPs in magnetic fluid (mg/mL).

The ISM assumes that the sample temperature is linearly increase during heating and that heat losses are negligible during the time interval used. Given that some researchers report SAR values in W per mass of particle [35-37] instead of per mass of iron [38-40], the calculations in this paper were performed using both units.

The CSM considers linear losses of the system due to a non-adiabatic setup. The formula below was used to calculate SAR [12]:

$${SAR}_{C S M} = \frac{( C \frac{Δ T}{Δ t} + L Δ T )}{{m}_{F e / MNPs}} , \qquad ( 3 )$$

where L is the linear loss parameter (mW/K).

The L was calculated in Excel (Microsoft Office) using the open-source corrected slope data analysis algorithm [41]. This algorithm estimates L by finding the value that results in the smallest standard deviation between SAR values on the same heating curve (30 second intervals, 5 fits).

In the phenomenological BLM, the heating curve is fitted using the following expression:

ΔT=a(1−e^{(-b(t-t}^{₀}⁾⁾). (4)

SAR values were calculated using the fitting parameters a and b as

$${SAR}_{B L M} = \frac{a b C}{{m}_{F e / MNPs}} . \qquad ( 5 )$$

The linear loss parameter L (W/K) for an individual sample was calculated as

L=bC. (6)

The intrinsic loss power parameter (ILP, nH·m²/kg) was proposed for normalization of a heating efficiency measured at different magnetic field amplitudes/frequencies [12]:

$$ILP = \frac{SAR}{{H}^{2} {f}^{′}} \qquad ( 7 )$$

where H is the field strength (kA/m);

ƒ is the frequency (kHz).

It should be noted that the ILP is limited to measurements for MNP samples with a dispersity greater than 0.1 in applied fields below the saturation field for the MNPs, and field frequencies between 10⁵-10⁶ Hz [32]. Additionally, the thermodynamic losses should not be greater than the power input from the field [42].

Iron Quantification. The iron content in each sample was quantified in triplicate using a colorimetric method adapted from the American Society of Testing and Materials (ASTM) standard method E394-15 for detection of trace amounts of iron [43]. Ten to 50 μL of each sample (depending on sample concentration) was digested for 12 hours in 1 mL of 70% HNO₃ at 101° C. to remove any coatings that may have been present. Ten to 30 μL of the acid solution with digested particles was evaporated for an hour at 115° C., followed by the addition of 46 μL of deionized water and 30 μL of hydroxylamine hydrochloride (8.06 M) to each sample at room temperature. The solution was vortexed and left to react for 1 hour to reduce the Fe(III) to Fe(II). After the reduction step, 49 μL of 1.22 M sodium acetate were added to each sample to buffer the solutions. Fe (II) cations were chelated by adding 75 μL of 13 mM 1,10-phenanthroline to form a colored complex (tris(1,10-phenanthroline) iron (II)) which have a maximum absorbance at 508 nm (ε=14,332±4,594 L/(mol·cm) at pH 4.8) (see Figure S2). Absorbance of the solutions was measured using SpectraMax M5 (Molecular Devices) microplate reader and 96 well plate (PN 9017, Corning) in a pathcheck mode. Iron concentration of samples was calculated using a standard calibration curve. Standard solutions of known iron concentration were prepared using an ICP iron standard in the concentration range from 0 to 5 μg/mL.

Dynamic light scattering (DLS). Ultraviolet-visible (UV-vis) spectroscopy was used to determine the optimal concentration of MNP dispersions for DLS measurements. A SpectraMax M5 (Molecular Devices) microplate reader equipped with a 24 well microplate and a 0.5 mm cover glass (Molecular Devices, SpectraDrop micro-volume starter kit, PN 0200-6262) was used to record UV-vis spectra. A calibration curve for each sample at 658 nm (the wavelength of the DLS laser) was made and used to estimate the optimal concentration of MNPs for DLS. A practical criterion is that the absorbance for a 1 cm cuvette must be smaller than 0.04 to exclude the effect of multiple scattering [44]. Using this criterion, the upper concentration threshold was determined for each type of MNP from the calibration curves (see Table SI), and this concentration was used for DLS measurements.

ZetaPals (Brookhaven Instruments Corporation) equipped with a 658 nm laser (35 mW) was used to determine particle size distribution (PSD, intensity-weighted) and hydrodynamic diameters of aqueous MNP dispersions. Each sample (100 μL) was measured in a plastic cuvette at 90° in triplicate (10 runs, 30 s) with the dust cutoff filter set to 40.00.

Intensity-weighted size distribution obtained from combined runs or its first peak in case of multimodal distribution was fitted to a lognormal function:

$$f ( d ) = \frac{A}{\sqrt{2 π} d σ} {e}^{- \frac{{( ln d - ln {d}_{s} )}^{2}}{2 {σ}^{2}}} , \qquad ( 8 )$$

where A is the area under the curve;

dₛ is the scale parameter;

σ is the shape parameter.

The arithmetic mean (d^{arithm.mean}), median (d^{median}), and mode (d^{mode}) diameters; variance (var(d)); and dispersity (Ð) were calculated from parameters of lognormal function according to the formulas below [45]:

$$\begin{matrix}{d}^{arithm . mean} = {d}_{s} \cdot {e}^{0.5 {σ}^{2}} , & ( 9 ) \\ {d}^{median} = {d}_{s} , & ( 10 ) \\ {d}^{mode} = {d}_{s} \cdot {e}^{- {σ}^{2}} , \\ & ( 11 ) \\ var ( d ) = {e}^{{σ}^{2}} ( {e}^{{σ}^{2}} - 1 ) {d}_{s}^{2} , & ( 12 ) \\ Ð = \frac{var ( d )}{{d}^{{mode}^{2}}} . & ( 13 )\end{matrix}$$

Given the non-symmetrical nature of pore size distribution (skewness), the mode diameter (d^{mode}) was used for the size characterization of the MNPs.

Nanoparticle tracking analysis (NTA). A NanoSight LM14 (Malvern Instruments Ltd.) equipped with a 405 nm laser (max. power 5 W) was used to determine the number-weighted particle size distributions, hydrodynamic diameters, and estimate nanoparticle concentrations (particles/mL) of MNPs dispersions. Samples at a concentration of 10 μg MNP/mL in distilled water were measured at least in triplicate. For each type of MNP, three video files (30 fps) with a duration of 60 s were captured and analyzed using NanoSight NTA 2.3 analytical software. The data obtained by NTA was analyzed using the same methodology as described in DLS section 2.4.

Vibrating sample magnetometry (VSM). For measurements of magnetic properties, 0.1 mg of MNPs were immobilized into agarose films. Briefly, 2% (w/v) agarose solution was prepared by dissolving agarose in distilled water upon heating with simultaneous mixing. After being completely dissolved, 500 μL of agarose solution was cooled down to approximately 50° C. and vortexed with 0.1 mg of MNPs. The agarose solution with MNPs was then transferred into a 48 well-plate (PN 3548, Costar) and dried overnight at 50° C.

For VSM measurements, films were fixed on 8 mm Pyrex sample holder (PN 1600-SHP, MicroSense) with double-sided carbon mounting tape. Magnetization of MNPs was measured in magnetic fields with strength from −1 T to 1 T (GMW Associates, Magnet Systems Model 3473-70 Electromagnet). Data was recorded using EasyVSM software. Measurements were performed once per film, while each magnetic field point was measured 4 times per measurement. The magnetization of blank agarose gel samples was measured in triplicate and used for the slope correction by its subtraction from the magnetization sample curves. Obtained data was plotted as mass magnetization (emu/(g particle)) vs. magnetic field (T) using OriginPro software. For comparison, mass magnetization of MNPs at 1 T (average of four points) was used.

Magnetic diameter analysis. The magnetic diameter was calculated by fitting the obtained magnetization vs. field curves (VSM data) to the Langevin-Chantrell function [46, 47] using Igor Pro 6.37 software (WaveMetrics):

$$\begin{matrix}M ( α ) = {M}_{s} {\int }_{0}^{∞} L ( α ) {n}_{v}^{*} ( {D}_{m} ) d {D}_{m} + x , & ( 14 ) \\ {n}_{v}^{*} ( {D}_{m} ) = \frac{1}{\sqrt{2 π} {D}_{m} ln σ} exp ( - \frac{{ln}^{2} ( \frac{{D}_{m}}{{D}_{mv}} )}{2 {ln}^{2} σ} ) , & ( 15 ) \\ \frac{M}{{M}_{s}} = L ( α ) = coth ( α ) - \frac{1}{α} , & ( 16 ) \\ α = \frac{π {μ}_{0} {M}_{d} {D}_{m}^{3} H}{6 {k}_{B} T} , & ( 17 ) \\ {M}_{d} = \frac{{M}_{s}}{φ} , & ( 18 )\end{matrix}$$

where α is the Langevin parameter (ratio of magnetic to thermal energy);
M_{S} is the saturation magnetization of MNPs in a nonmagnetic medium (A/m);
n*ᵥ(Dₘ) is the normalized volume distribution of particles (nᵥ(Dₘ)/∫₀^{∞}nᵥ(Dₘ)dDₘ);
Dₘ is the magnetic diameter (m);
x is the high field slope, equal to the product of background susceptibility and applied magnetic field (A/m);
ln σ is the shape parameter (and is the standard deviation of the log of the distribution);
Dₘᵥ is the volume-weighted median magnetic diameter (m);
μ₀ is the magnetic permeability of free space (4π·10⁻⁷ H/m²);
M_{d} is the domain magnetization of the MNPs (A/m);
H is the applied magnetic field (A/m);
k_{B} is Boltzmann's constant (1.38·10⁻²³ J/K);
T is the temperature (K);
φ is the volume fraction of MNPs in the sample.

The domain magnetization for magnetite was assumed to be equal to that of bulk magnetite (446 kA/m) [48]. Temperature (T, 295 K) and domain magnetization (M_{d}) were held constant, while values for volume fraction (φ), magnetic diameter (Dₘᵥ), shape parameter (ln σ), and high field slope (x) were adjusted.

To convert the geometric mean magnetic diameter (Dₘᵥ^{geometric}) and ln σ obtained from the Langevin-Chantrell fit into the arithmetic mean magnetic diameter (Dₘᵥ^{arithm.mean}) and standard deviation, the following formulas were used:

Dₘᵥ^{arithm.mean}=exp(ln Dₘᵥ^{geometric}+½ ln² σ), (19)

std.dev=Dₘᵥ^{arithm.mean}\sqrt{exp(ln² σ)−1.} (20)

Transmission electron microscopy (TEM). For TEM analysis, particle samples were placed on a copper grid with a carbon supporting film. A FEI Tecnai F20 S/TEM microscope with a Schottky field emission ZrO/W gun source was used to image the following samples: Micromod 79-01-102 and Sigma-Aldrich 747335 (lots MKBW1867V and MKCD1194), 900028, and 900042. ImageJ software (version 1.51j) was used to measure the core diameter of the particles (n=100) from TEM images. For the rest MNPs, the TEM diameter provided by the manufacturer was used for calculation of the magnetically dead layer thickness (D_{mdl}). The magnetically dead layer is a surface layer of the magnetic particle in which magnetic dipoles are disordered and magnetic properties are degraded [46, 49, 50]. The thickness of the magnetically dead layer was calculated as

$${D}_{mdl} = \frac{{D}_{TEM} - {D}_{m v}^{a rithm . mean}}{2} , \qquad ( 21 )$$

where D_{TEM} is the core diameter obtained from TEM images.

Statistical data analysis. The data is presented as an arithmetic mean±standard uncertainty of the mean (s(Y̅)) [51]. The s(Y̅) was estimated as:

$$s ( \overline{Y} ) = \sqrt{{( \frac{s}{\sqrt{N}} {t}_{68.27} ( v ) )}^{2} + {( Δ y )}^{2}} , \qquad ( 22 )$$

where s is the sample standard deviation;
N is the number of parallel experiments;
tₚ(ν) is the critical value for p percent of two-tailed t-distribution with defined degrees of freedom (ν);
Δy is the error of indirect measurements, which was calculated using the numerical differentiation method [52]:

$$Δ y = {\sum }_{i = 1}^{n} \vert \frac{\partial f}{\partial {x}_{i}} \vert Δ {x}_{i} , \qquad ( 23 )$$

where ƒ is a function of directly measured variables xᵢ;
Δxᵢ is the error of the direct measurements.

Systematic error was considered negligible for all measurements.

Differences between groups were examined for statistical significance using two-tailed paired t-test or one-way ANOVA for a significance level α=0.05. The paired t-test was used to compare differences between ILP values for particles obtained at 345 kHz/74 mT and 223 kHz/51 mT. The one-way ANOVA was used to compare the three calculation methods (initial slope, corrected slope, and Box-Lucas methods). As per recommendation by National Institute of Standards and Technology (NIST), both the Bonferroni and Tukey's tests were used as post-hoc tests for one-way ANOVA, and the smallest confidence band was selected [53].

To evaluate correlations between SAR (calculated by the Box-Lucas method at 345 kHz/74 mT) and other parameters (iron content, hydrodynamic diameter, magnetic diameter, core diameter, magnetically dead layer thickness, and saturation mass magnetization), the Pearson's product-moment correlation coefficient and Spearman's rank correlation coefficient were calculated. The latter one is the nonparametric version of the Pearson product-moment correlation. Both coefficients measure the strength and direction of association between two ranked variables. However, Spearman's correlation determines the strength and direction of the monotonic relationship between the two variables rather than the strength and direction of the linear relationship between the variables, which is what Pearson's correlation does.

All calculations were performed using Microsoft Excel 2016, while Minitab 18 and OriginPro 2018 (b9.5.0.193) were used for statistical analysis, nonlinear and linear fitting, plotting graphs, and graphical residual analysis.

### Results:

Table 3.1 shows an overview of data collected on the characterization of commercially available SPIONs. In addition to this, SAR was calculated per iron concentration. ILP values were also calculated, but are not shown. SAR was also measured at 223 kHz and 26 kA/m, and at concentrations of 5 mg/ml and 10 mg/ml for SPIONs that came in more concentrated aqueous solutions than 1 mg/ml. Detailed calculations for all of this data are provided in the article referenced above. UV-Vis measurements to determine the maximum concentration for use in DLS were measured, and DLS measurements were performed.

TABLE 3.1
Characterization of commercially available SPIONs. Nanoparticle tracking analysis (NTA) and dynamic light scattering
(DLS) was performed and iron concentration was quantified using a UV-Vis method based on o-phenanthroline complexes.
SAR values were taken for 200 μL of sample (1 mg/ml) measured at 59 kA/m and 345 kHz in the Ambrell Easyheat.
SAR was calculated using the Box-Lucas method. Values reported as average ± standard error.

| | | Iron Content | SAR_{Box-Lucas} | | Hydrodynamic diameter | |
|---|---|---|---|---|---|---|
| Company | Product Number | (μg Fe/mg | (W/g MNPs) | (W/g Fe) | from DLS (nm) | from NTA (nm) |
| Chemicell | Fluidmag/C11-D | 386.2 ± 35.7 | 217.4 ± 56.6 | 562.8 ± 198.6 | 81.5 ± 11.9 | 80.8 ± 1.6 |
| Imagion | —COOH func. Fe₃O₄ | 686.1 ± 40.4 | 538.1 ± 43.2 | 784.3 ± 109.2 | 35.0 ± 1.7 | 38.0 ± 2.8 |
| Biosystems | mPEG coated Fe₃O₄ | 762.4 ± 93.1 | 639.8 ± 52.3 | 839.2 ± 171.1 | 66.8 ± 8.1 | 103.4 ± 15.5 |
| Micromod | 09-56-132 | 530.7 ± 124.5 | 190.5 ± 47.2 | 359.0 ± 173.1 | 210.4 ± 10.5 | 87.4 ± 3.4 |
| | 09-56-252 | 479.8 ± 49.6 | 178.6 ± 6.2 | 372.4 ± 51.3 | 303.7 ± 4.8 | 109.8 ± 10.4 |
| | 102-00-132 | 350.2 ± 11.5 | 322.7 ± 76.3 | 921.5 ± 248.0 | 62.9 ± 1.2 | 115.1 ± 22.6 |
| | 104-00-501 | 470.4 ± 20.9 | 552.9 ± 73.9 | 1175.4 ± 209.4 | 54.5 ± 1.8 | 94.7 ± 9.5 |
| | 79-01-102 | 424.6 ± 25.0 | 95.5 ± 13.8 | 224.9 ± 45.7 | 50.0 ± 8.0 | 44.7 ± 4.9 |
| nanoTherics | HyperMAG A | 263.1 ± 15.7 | 492.5 ± 47.3 | 1811.1 ± 291.7 | 191.0 ± 35.2 | 101.8 ± 9.2 |
| | HyperMAG B | 705.4 ± 16.2 | 710.1 ± 29.2 | 1006.7 ± 64.5 | 150.3 ± 21.4 | 117.0 ± 13.9 |
| | HyperMAG C | 740.7 ± 22.4 | 491.6 ± 60.0 | 663.6 ± 101.0 | 258.6 ± 59.3 | 77.9 ± 21.5 |
| Ocean | SEI-10-05 | 689.0 ± 70.0 | 166.5 ± 18.6 | 241.6 ± 51.6 | 71.5 ± 7.0 | 77.9 ± 2.0 |
| NanoTech | SHA-10-02 | 600.5 ± 28.2 | 156.9 ± 31.2 | 261.4 ± 64.3 | 35.1 ± 2.3 | 44.8 ± 18.2 |
| | SPP-05-02 | 914.2 ± 63.7 | 89.1 ± 28.8 | 97.5 ± 38.3 | 38.4 ± 2.0 | 33.4 ± 2.7 |
| Sigma- | 544884^{a} | 279.9 ± 48.3 | 657.1 ± 158.1 | 2347.5 ± 969.7 | 123.7 ± 11.5 | 96.7 ± 10.0 |
| Aldrich | 637149^{a} | 47.65 ± 0.06 | 81.6 ± 23.7 | 1712.5 ± 499.5 | n/a, not stable | n/a, not stable |
| | 725331 | 244.7 ± 10.9 | 49.5 ± 5.1 | 202.3 ± 29.8 | 59.5 ± 18.0 | 65.3 ± 6.8 |
| | 747254 | 773.7 ± 45.8 | 237.8 ± 74.4 | 307.4 ± 114.4 | 50.4 ± 8.2 | 125.3 ± 2.1 |
| | 747335^{b} | 722.8 ± 71.3 | 1040.8 ± 35.2 | 1439.9 ± 190.6 | 46.4 ± 8.4 | 63.4 ± 7.0 |
| | 747335^{c} | 730.8 ± 42.3 | 952.1 ± 72.3 | 1302.8 ± 174.2 | 57.1 ± 12.3 | 77.6 ± 0.6 |
| | 747440 | 10.6 ± 1.6 | 46.1 ± 6.7 | 4349.6 ± 1292.5 | 32.9 ± 5.0 | 51.9 ± 1.1 |
| | 747459 | 7.9 ± 0.8 | 68.9 ± 16.1 | 8721.5 ± 2921.2 | 30.4 ± 2.8 | 41.2 ± 30.8 |
| | 747467 | 7.2 ± 0.8 | 54.5 ± 5.5 | 7569.4 ± 1604.9 | 44.0 ± 4.5 | 49.9 ± 3.0 |
| | 773352^{a} | 47.60 ± 0.06 | 79.2 ± 12.6 | 1664.5 ± 267.2 | n/a, not stable | 44.1 ± 9.3 |
| | 900026 | 1008.2 ± 46.0 | 330.4 ± 102.4 | 327.7 ± 116.6 | 31.3 ± 4.3 | 48.4 ± 4.3 |
| | 900027 | 783.5 ± 22.2 | 408.5 ± 31.0 | 521.5 ± 54.4 | 61.3 ± 6.4 | 51.4 ± 2.9 |
| | 900028 | 645.3 ± 41.3 | 1371.4 ± 41.8 | 2125.3 ± 200.8 | 47.2 ± 6.6 | 66.7 ± 8.5 |
| | 900042 | 977.8 ± 55.4 | 2158.4 ± 110.1 | 2207.3 ± 237.7 | 45.7 ± 0.7 | 41.0 ± 4.7 |
| | 900043 | 862.1 ± 15.3 | 354.8 ± 47.3 | 411.6 ± 62.2 | 28.6 ± 1.3 | 27.7 ± 8.0 |
| | 900062 | 654.0 ± 69.4 | 832.7 ± 69.7 | 1273.3 ± 241.6 | 49.6 ± 3.7 | 58.9 ± 0.6 |
| | 900146 | 293.4 ± 26.4 | 118.9 ± 11.1 | 405.3 ± 74.2 | 32.5 ± 0.4 | 55.1 ± 5.5 |
| n/a | Water control | 0 | 135.9 ± 11.7 | n/a | n/a | n/a |

It was found that the ranking of MNPs based on their SAR values marginally depends on magnetic field parameters and SAR units. Sigma-Aldrich 900042 MNPs have the highest BLM-SAR value: 2158.4±110.1 W/(g MNP) at 345 kHz/59 kA/m. These MNPs have an iron content of 977.8±55.4 μg Fe/(mg MNP), a magnetic diameter of 12.8±5.8 nm, a mass magnetization value of 90.7±4.5 emu/(g MNP) at 1 T, a core diameter of 22.9±0.2 nm, and a magnetically dead layer thickness equal to 5.1±2.9 nm. They can be heated by 36.9±2.0 K (see Table SII) in 120 s under AMF application (345 kHz/74 mT) at a concentration of 1 mg MNP/ml, indicating that they can be an optimal candidate for MFH treatment.

### Discussion:

Determined SAR values for MNPs often vary between labs due to poor standardization of methodology and measurement conditions. Therefore, the goal of this study was to compare a wide variety of MNPs under the same conditions. In addition, companies often do not provide detailed information on physicochemical, colloidal, and magnetic properties of MNPs, with SAR, in particular, being rarely reported. Thus, an extensive evaluation and characterization of 31 commercial MNPs including SAR (measured at two different AMFs), ILP, and physical and magnetic properties was performed to help to guide researchers in identifying the optimal MNPs for their studies. As a result, the top MNPs identified in this study (such as illustrated in Table 3.1) were identified as great candidates for SAR applications. Among evaluated MNPs, Sigma-Aldrich 900042 MNPs have the highest BLM-SAR value equal to 2158.4±110.1 W/(g MNP) at 345 kHz/74 mT.

A comprehensive study of physical and magnetic properties of several commercial MNPs including calculation of SAR and ILP was performed. High-SAR MNPs such as Sigma-Aldrich 900042, 900028, 747335, 900062, and nanoTherics HyperMAG B were identified, and three SAR calculation methods (initial and corrected slope methods, and Box-Lucas method) were validated using a graphical residual analysis. Based on the results of model validation we suggest to other researchers to use non-linear Box-Lucas method for SAR calculation and express BLM-SAR values in both units: W/(g MNP) and W/(g Fe). Positive correlations (from moderate to strong) between SAR values and other MNP properties were found. Studied MNP properties, such as the magnetically dead layer thickness, might contribute to the nanoparticle anisotropy and could be responsible for efficient heating of MNP in AMF

### LITERATURE CITED

1. Carmeliet, P. & Jain, R. K. Molecular mechanisms and clinical applications of angiogenesis. Nature 473, 298-307 (2011).
2. Simons, M. et al. Clinical Trials in Coronary Angiogenesis: Issues, Problems, Consensus: An Expert Panel Summary. Circulation 102, e73-e86 (2000).
3. Moore, M. C., Pandolfi, V. & McFetridge, P. S. Novel human-derived extracellular matrix induces in vitro and in vivo vascularization and inhibits fibrosis. Biomaterials 49, 37-46 (2015).
4. Lloyd-Jones, D. et al. Heart Disease and Stroke Statistics—2010 Update. Circulation 121, (2010).
5. Musculoskeletal injuries report: incidence, risk factors, and prevention. (2000).
6. William Axelrad, T., Kakar, S. & Einhorn, T. A. New technologies for the enhancement of skeletal repair. Injury 38, S49-S62 (2007).
7. Robich, M. P., Chu, L. M., Oyamada, S., Sodha, N. R. & Sellke, F. W. Myocardial therapeutic angiogenesis: a review of the state of development and future obstacles. Expert Rev. Cardiovasc. Ther. 9, 1469-79 (2011).
8. Gómez-Barrena, E. et al. Bone fracture healing: Cell therapy in delayed unions and nonunions. Bone 70, 93-101 (2015).
9. Eming, S. A., Martin, P. & Tomic-Canic, M. Wound repair and regeneration: Mechanisms, signaling, and translation. Sci. Transl. Med. 6, (2014).
10. Barrientos, S., Brem, H., Stojadinovic, O. & Tomic-Canic, M. Clinical application of growth factors and cytokines in wound healing. Wound Repair Regen. 22, 569-78 (2014).
11. Richardson, T. P., Peters, M. C., Ennett, A. B. & Mooney, D. J. Polymeric system for dual growth factor delivery. Nat. Biotechnol. Publ. online 1 Nov. 2001; |doi10.1038/nbt1101-1029 19, 1029 (2001).
12. Saik, J. E., Gould, D. J., Watkins, E. M., Dickinson, M. E. & West, J. L. Covalently immobilized platelet-derived growth factor-BB promotes angiogenesis in biomimetic poly(ethylene glycol) hydrogels. Acta Biomater. 7, 133-143 (2011).
13. Lazarous, D. F. et al. Adenoviral-mediated gene transfer induces sustained pericardial VEGF expression in dogs: effect on myocardial angiogenesis. Cardiovasc. Res. 44, 294-302 (1999).
14. Khan, T. A., Sellke, F. W. & Laham, R. J. Gene therapy progress and prospects: therapeutic angiogenesis for limb and myocardial ischemia. Gene Ther. 10, 285-291 (2003).
15. Losordo, D. W. & Dimmeler, S. Therapeutic Angiogenesis and Vasculogenesis for Ischemic Disease: Part II: Cell-Based Therapies. Circulation 109, 2692-2697 (2004).
16. Kawamoto, A., Asahara, T. & Losordo, D. W. Transplantation of endothelial progenitor cells for therapeutic neovascularization. Cardiovasc. Radiat. Med. 3, 221-5 (2002).
17. Suarez, Y. & Sessa, W. C. MicroRNAs As Novel Regulators of Angiogenesis. Circ. Res. 104, 442-454 (2009).
18. Tonello, S., Moore, M. C., Sharma, B., Dobson, J. & McFetridge, P. S. Controlled release of a heterogeneous human placental matrix from PLGA microparticles to modulate angiogenesis. Drug Deliv. Transl. Res. 6, 174-183 (2016).
19. White, A. P. et al. Clinical applications of BMP-7/OP-1 in fractures, nonunions and spinal fusion. Int. Orthop. 31, 735-741 (2007).
20. Timko, B. P., Dvir, T. & Kohane, D. S. Remotely triggerable drug delivery systems. Adv. Mater. 22, 4925-43 (2010).
21. Fomina, N., Sankaranarayanan, J. & Almutairi, A. Photochemical mechanisms of light-triggered release from nanocarriers. Adv. Drug Deliv. Rev. 64, 1005-20 (2012).
22. Kost, J. & Langer, R. Responsive polymeric delivery systems. Adv. Drug Deliv. Rev. 46, 125-148 (2001).
23. Pankhurst, Q. A., Connolly, J., Jones, S. K. & Dobson, J. Applications of magnetic nanoparticles in biomedicine. 36, (2003).
24. Kim, J. E., Shin, J. Y. & Cho, M. H. Magnetic nanoparticles: An update of application for drug delivery and possible toxic effects. Arch. Toxicol. 86, 685-700 (2012).
25. Ratner, B. D. & Hoffman, A. S. Biomaterials Science: An Introduction to Materials in Medicine. (Academic Press, 2012). doi:10.1016/B978-0-08-087780-8.00025-5
26. Sirivisoot, S. & Harrison, B. S. Magnetically stimulated ciprofloxacin release from polymeric microspheres entrapping iron oxide nanoparticles. Int. J. Nanomedicine 10, 4447-58 (2015).
27. Jameela, S. R. et al. Poly (e-caprolactone) microspheres as a vaccine carrier. Curr. Sci. 70, 669-671 (1996).
28. Cao, X. & Shoichet, M. S. Delivering neuroactive molecules from biodegradable microspheres for application in central nervous system disorders. Biomaterials 20, 329-339 (1999).
29. Stanković, M. et al. Tailored protein release from biodegradable poly(ε-caprolactone-PEG)-b-poly(ε-caprolactone) multiblock-copolymer implants. Eur. J. Pharm. Biopharm. 87, 329-337 (2014).
30. Woodruff, M. A. & Hutmacher, D. W. The return of a forgotten polymer—Polycaprolactone in the 21st century. Prog. Polym. Sci. 35, 1217-1256 (2010).
31. Pitt, C. G., Jeffcoat, A. R., Zweidinger, R. A. & Schindler, A. Sustained Drug Delivery Systems. I. The Permeability of Poly(e-caprolactone), Poly(DL-lactic acid), and their copolymers. J. Biomed. Mater. 13, 497-507 (1979).
32. Sinha, V. R., Bansal, K., Kaushik, R., Kumria, R. & Trehan, A. Poly-ϵ-caprolactone microspheres and nanospheres: an overview. Int. J. Pharm. 278, 1-23 (2004).
33. Coey, J. M. D. Magnetism and Magnetic Materials. (Cambridge University Press, 2009). doi:10.1016/0160-9327(88)90178-0
34. Brown, W. F. Thermal fluctuations of a single-domain particle. Phys. Rev. 130, 1677-1686 (1963).
35. Hofmann-Amtenbrink, M., Von Rechenberg, B. & Hofmann, H. in Nanostructured materials for biomedical applications (ed. Tan, M. C.) 695-23 (2009).
36. Mahmoudi, M., Sant, S., Wang, B., Laurent, S. & Sen, T. Superparamagnetic iron oxide nanoparticles (SPIONs): Development, surface modification and applications in chemotherapy. Adv. Drug Deliv. Rev. 63, 24-46 (2011).
37. Pouliquen, D., Le Jeune, J. J., Perdrisot, R., Ermias, A. & Jallet, P. Iron oxide nanoparticles for use as an MRI contrast agent: Pharmacokinetics and metabolism. Magn. Reson. Imaging 9, 275-283 (1991).
38. Lubbe, A. S., Alexiou, C. & Bergemann, C. Clinical Applications of Magnetic Drug Targeting. J. Surg. Res. 95, 200-206 (2001).
39. Luo, S. et al. Clinical trials of magnetic induction hyperthermia for treatment of tumours. OA Cancer. OA Cancer 2, 2 (2014).
40. Ahmed, M. et al. Magnetic sentinel node and occult lesion localization in breast cancer (MagSNOLL Trial). Br. J. Surg. 102, 646-652 (2015).
41. Dobson, J. Remote control of cellular behaviour with magnetic nanoparticles. Nat. Nanotechnol. 3, 139-143 (2008).
42. Dobson, J. Gene therapy progress and prospects: magnetic nanoparticle-based gene delivery. Gene Ther. 13, 283-287 (2006).
43. Kang, J. H. et al. An extracorporeal blood-cleansing device for sepsis therapy. Nat. Med. 20, 1211-1216 (2014).
44. Zhao, X. et al. Active scaffolds for on-demand drug and cell delivery. Proc. Natl. Acad. Sci. U.S.A. 108, 67-72 (2011).
45. Yoo, D., Lee, J.-H., Shin, T.-H. & Cheon, J. Theranostic Magnetic Nanoparticles. Acc. Chem. Res. 44, 863-874 (2011).
46. Rosensweig, R. E. Heating magnetic fluid with alternating magnetic field. J. Magn. Magn. Mater. 252, 370-374 (2002).
47. Kallumadil, M. et al. Suitability of commercial colloids for magnetic hyperthermia. J. Magn. Magn. Mater. 321, 1509-1513 (2009).
48. Wildeboer, R. R., Southern, P. & Pankhurst, Q. A. On the reliable measurement of specific absorption rates and intrinsic loss parameters in magnetic hyperthermia materials. J. Phys. D. Appl. Phys. 47, 14 (2014).
49. Bird, R. B., Stewart, W. E. & Lightfoot, E. N. Transport Phenomena. Applied Mechanics Reviews 55, (2002).
50. Einstein, A. Investigations on the Theory of the Brownian Movement. Annalen der Physik 19, 579 (1905).
51. Stokes, G. On the effect of the internal friction of fluids on the motion of pendulums. Trans. Cambridge Philos. Soc. 9, (1950).
52. Kim, Y.-C. & Myerson, A. S. Diffusivity of Protein in Aqueous Solutions. Korean J. CheJ˜L Eng 13, 288-293 (1996).
53. Young, D. F., Munson, B. R., Okiishi, T. H. & Huebsch, W. W. A brief introduction to fluid mechanics. (John Wiley & Sons, Inc., 2007).
54. Ramanath, H. S., Chua, C. K., Leong, K. F. & Shah, K. D. Melt flow behaviour of poly-ε-caprolactone in fused deposition modelling. J. Mater. Sci. Mater. Med. 19, 2541-2550 (2008).
55. Sakellari, D., Mathioudaki, S., Kalpaxidou, Z. & Simeonidis, K. Exploring multifunctional potential of commercial ferrofluids by magnetic particle hyperthermia. J. Magn. Magn. Mater. 380, 360-364 (2015).
56. Pankhurst, Q. A., Thanh, N. K. T., Jones, S. K. & Dobson, J. Progress in applications of magnetic nanoparticles in biomedicine. J. Phys. D. Appl. Phys. 42, 1-15 (2009).
57. Ceylan, S., Coutable, L., Wegner, J. & Kirschning, A. Inductive heating with magnetic materials inside flow reactors. Chem.—A Eur. J. 17, 1884-1893 (2011).
58. Ibraheem, D., Iqbal, M., Agusti, G., Fessi, H. & Elaissari, A. Effects of process parameters on the colloidal properties of polycaprolactone microparticles prepared by double emulsion like process. Colloids Surfaces A Physicochem. Eng. Asp. 445, 79-91 (2014).
59. Shenoy, D. B., D'Souza, R. J., Tiwari, S. B. & Udupa, N. Potential Applications of Polymeric Microsphere Suspension as Subcutaneous Depot for Insulin. Drug Dev. Ind. Pharm. 29, 555-563 (2003).
60. Bordes, C. et al. Determination of poly(s-caprolactone) solubility parameters: Application to solvent substitution in a microencapsulation process. Int. J. Pharm. 383, 236-243 (2010).
61. Hsu, K. H. et al. Hybrid Electrospun Polycaprolactone Mats Consisting of Nanofibers and Microbeads for Extended Release of Dexamethasone. Pharm. Res. 33, 1509-1516 (2016).
62. D'Souza, S. S., Faraj, J. A. & DeLuca, P. P. A model-dependent approach to correlate accelerated with real-time release from biodegradable microspheres. AAPS PharmSciTech 6, E553-E564 (2005).
63. Bengani, L. C., Leclerc, J. & Chauhan, A. Lysozyme transport in p-HEMA hydrogel contact lenses. J. Colloid Interface Sci. 386, 441-450 (2012).
64. Rothstein, S. N., Federspiel, J. & Little, S. R. A simple model framework for the prediction of controlled release from bulk eroding polymer matrices. 1873-1880 (2008). doi:10.1039/b718277e
65. Arifin, D. Y., Lee, L. Y. & Wang, C.-H. Mathematical modeling and simulation of drug release from microspheres: Implications to drug delivery systems. Adv. Drug Deliv. Rev. 58, 1274-1325 (2006).
66. Siepmann, J. & Göpferich, A. Mathematical modeling of bioerodible, polymeric drug delivery systems. Adv. Drug Deliv. Rev. 48, 229-247 (2001).
67. Rothstein, S. N., Federspiel, W. J. & Little, S. R. A unified mathematical model for the prediction of controlled release from surface and bulk eroding polymer matrices. Biomaterials 30, 1657-64 (2009).
68. Siegel, R. A. & Langer, R. Mechanistic studies of macromolecular drug release from macroporous polymers. II. Models for the slow kinetics of drug release. J. Control. Release 14, 153-167 (1990).
69. Siepmann, J., Kranz, H., Bodmeier, R. & Peppas, N. A. HPMC-Matrices for Controlled Drug Delivery: A New Model Combining Diffusion, Swelling, and Dissolution Mechanisms and Predicting the Release Kinetics. Pharm. Res. 16, 1748-1756 (1999).
70. Siepmann, J. & Peppas, N. A. Hydrophilic Matrices for Controlled Drug Delivery: An Improved Mathematical Model to Predict the Resulting Drug Release Kinetics (the ‘sequential Layer’ Model). Pharm. Res. 17, 1290-1298 (2000).
71. Davidson, A. M. Magnetic Induction Heating of Superparamagnetic Nanoparticles for Applications in the Energy Industry.
72. Peppas, N. A. Analysis of Fickian and non-Fickian drug release from polymers. Pharm. Acta Helv. 60, 110-1 (1985).
73. Siepmann, J. & Peppas, N. A. Higuchi equation: Derivation, applications, use and misuse. Int. J. Pharm. 418, 6-12 (2011).
74. Park, K. Controlled drug delivery systems: Past forward and future back. J. Control. Release 190, 3-8 (2014).
75. Purcell, B. P. et al. Injectable and bioresponsive hydrogels for on-demand matrix metalloproteinase inhibition. Nat. Mater. 13, 653-661 (2014).
76. Hsieh, D. S. T., Langer, R. & Folkman, J. Magnetic modulation of release of macromolecules from polymers. Med. Sci. 78, 1863-1867 (1981).
77. Edelman, E. R., Kost, J., Bobeck, H. & Langer, R. Regulation of drug release from polymer matrices by oscillating magnetic fields. J. Biomed. Mater. Res. 19, 67-83 (1985).
78. Kost, J., Wofrum, J. & Langer, R. Magnetically enhanced isulin release in diabetic rats. J. Biomed. Mater. 21, 1367-1373 (1987).
79. Kost, J., Noecker, R., Kunica, E. & Langer, R. Magnetically Controlled Release Systems—Effect of Polymer Composition. J. Biomed. Mater. Res. 19, 935-940 (1985).
80. Saslawski, 0, Weingarten, C., Benoit, J.-P. & Couvreur, P. Magnetically responsive microspheres for the pulsed delivery of insulin. Life Sci. 42, 1521-1528 (1988).
81. Booth, A., Pintre, I. C., Lin, Y., Gough, J. E. & Webb, S. J. Release of proteins and enzymes from vesicular compartments by alternating magnetic fields. Phys. Chem. Chem. Phys. 17, 15579-15588 (2015).
82. Guilherme, M. R. et al. Albumin release from a brain-resembling superabsorbent magnetic hydrogel based on starch. Soft Matter 8, 6629 (2012).
83. Finotelli, P. V. et al. Microcapsules of alginate/chitosan containing magnetic nanoparticles for controlled release of insulin. Colloids Surfaces B Biointerfaces 81, 206-211 (2010).
84. Chen, J.-P. et al. Magnetically controlled release of recombinant tissue plasminogen activator from chitosan nanocomposites for targeted thrombolysis. J. Mater. Chem. B 4, 2578-2590 (2016).
85. Honarmand, D., Ghoreishi, S. M., Habibi, N. & Nicknejad, E. T. Controlled release of protein from magnetite-chitosan nanoparticles exposed to an alternating magnetic field. J. Appl. Polym. Sci. 133, n/a-n/a (2016).
86. Zheng, C., Ding, Y., Liu, X., Wu, Y. & Ge, L. Highly magneto-responsive multilayer microcapsules for controlled release of insulin. Int. J. Pharm. 475, 17-24 (2014).
87. Baeza, A., Guisasola, E., Ruiz-Hernández, E. & Vallet-Regí, M. Magnetically Triggered Multidrug Release by Hybrid Mesoporous Silica Nanoparticles. Chem. Mater. 24, 517-524 (2012).
88. Kim, H., Park, H., Lee, J. W. & Lee, K. Y. Magnetic field-responsive release of transforming growth factor beta 1 from heparin-modified alginate ferrogels. Carbohydr. Polym. 151, 467-473 (2016).

1. A composite microparticle comprising:
a plurality of magnetic nanoparticles;
a biologically active agent; and
a biocompatible polymer matrix comprising a polymer having a melting point higher than normal body temperature and lower than a deactivation temperature of the biologically active agent, wherein the magnetic nanoparticles and biologically active agent are at least partially encapsulated in the polymer matrix, such that application of an alternating magnetic field induces the magnetic nanoparticles to generate heat effective to melt the polymer matrix, thereby releasing the biologically active agent.

2. The composite microparticle of claim 1, wherein the magnetic nanoparticles comprise superparamagnetic iron oxide nanoparticles (SPIONs).

3. The composite microparticle of claim 2, wherein the SPIONs are selected from the group consisting of: Fe₃O₄ and γFe₂O₃.

4. The composite microparticle of claim 2, wherein the SPIONs have an average diameter of about 5 nm to 100 nm.

5. The composite microparticle of claim 1, wherein the biologically active agent comprises biomolecules selected from the group consisting of: proteins, antibodies, nucleotides, carbohydrates, lipids, and mixtures thereof.

6. (canceled)

7. The composite microparticle of claim 1, wherein the biologically active agent comprises human placental matrix (hPM).

8. The composite microparticle of claim 1, wherein the biologically active agent comprises small molecule therapeutic agents.

9. (canceled)

10. The composite microparticle of claim 1, wherein the polymer matrix comprises a biocompatible polymer having a melting point of about 38° C. or higher.

11. The composite microparticle of claim 1, wherein the polymer matrix comprises a biocompatible polymer having a melting point of about 42° C. to 60° C.

12. The composite microparticle of claim 1, wherein the polymer matrix comprises a biocompatible polymer selected from the group consisting of: polycaprolactone (PCL), polyvalerolactone (PVL), Poly(acrylic acid, butyl ester) aka PolyButyl Acrylate, PEG4000, and PEG6000, Polyisobutylene, PEG-Distearate MP, Agarose, Poly(azelaic anhydride), Poly(Ethylene adipate), and combinations thereof.

13. (canceled)

14. The composite microparticle of claim 1, wherein the microparticles comprise about 10 to 50% biologically active agent.

15. The composite microparticle of claim 1, wherein the microparticles comprise about 5 to 80% (w/w) magnetic nanoparticles.

16. (canceled)

17. The composite microparticle of claim 16, wherein the microparticles are lyophilized.

18. A system for targeted delivery and release of biologically active agents in vivo, the system comprising:
a plurality of composite microparticles of claim 1; and
an alternating current (AC) magnetic field generator, wherein the plurality of composite microparticles are adapted for delivery to a subject in need of treatment with the biologically active agent and wherein application of an AC magnetic field to a target area of the patient induces the magnetic nanoparticles to generate heat effective to melt the polymer matrix, thereby releasing the biologically active agent to the target area of the subject in vivo.

19. The system of claim 18, wherein the field generator has a field strength of about 5 to 50 kA/m.

20. The system of claim 18, wherein the field generator has a frequency of about 100 to 1,000 kHz.

21. (canceled)

22. A method for targeted delivery and release of a biologically active agent to a subject in need of treatment, the method comprising:
delivering to the subject in need of treatment with the biologically active agent a plurality of biocompatible composite microparticles each microparticle comprising a plurality of magnetic nanoparticles, a biologically active agent, and a biocompatible polymer matrix comprising a polymer having a melting point higher than normal body temperature and lower than a deactivation temperature of the biologically active agent, wherein the magnetic nanoparticles and biologically active agent are at least partially encapsulated in the polymer matrix;
applying a magnetic field to a target area of the subject to direct the microparticles to the target area; and
applying an AC magnetic field to the target area of the subject effective to induce the magnetic nanoparticles to generate heat effective to melt the polymer matrix, thereby releasing the biologically active agent to the target area of the subject.

23. The method of claim 22, wherein the AC magnetic field has a strength of about 5 to 37.5 kA/m.

24. The method of claim 22, wherein the AC magnetic field frequency of about 100 to 1,000 kHz.

25. The method of claim 22, wherein the AC magnetic field is applied for a time period of about 0.1 to 3 hours.

26-35. (canceled)
